(19) United States
(12) Patent Application Publication
(10) Pub. No.: US 2018/0346473 A1
(43) Pub. Date: Dec. 6, 2018

(54) FUSED BICYCLIC COMPOUNDS

(71) Applicant: Akarna Therapeutics, Ltd., Cambridge (GB)

(72) Inventors: Raju MOHAN, Encinitas, CA (US); Benjamin Anthony PRATT, Encinitas, CA (US)

(73) Assignee: Akarna Therapeutics, Ltd., Hsinchu (TW)

(21) Appl. No.: 15/994,222

(22) Filed: May 31, 2018

Related U.S. Application Data
(60) Provisional application No. 62/514,060, filed on Jun. 2, 2017.

(51) Int. Cl. C07D 487/04; C07D 265/30; C07D 231/12; C07F 3/06; C07D 231/16; C07C 69/738

(52) U.S. Cl. CPC C07D 487/04; C07D 265/30; C07C 69/738; C07F 3/06; C07D 231/16; C07D 231/12

(57) ABSTRACT

Described herein are fused bicyclic compounds, compositions, and methods for their preparation.

### BACKGROUND OF THE INVENTION

The present application claims the benefit of U.S. provisional application No. 62/514,060, filed Jun. 2, 2017, the contents of which are herein incorporated by reference in their entirety.

### BACKGROUND OF THE INVENTION

Farnesoid X receptor (FXR) is a member of the nuclear hormone receptor superfamily of ligand-activated transcription factors. Bile acids are FXR physiological ligands. On activation by bile acids, FXR regulates a wide variety of target genes that are critically involved in the control of bile acid, lipid and glucose homeostasis. Thus, FXR plays a key role in the pathogenesis of cholestatic diseases, non-alcoholic fatty liver disease and inflammatory bowel disease.

### SUMMARY OF THE INVENTION

Described herein are processes for the synthesis of FXR modulatators, wherein the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. Further described herein are pharmaceutical compositions comprising (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

In one aspect is a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1):

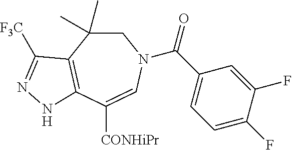

comprising contacting a compound with the structure:

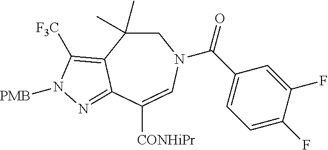

with acid followed by base.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

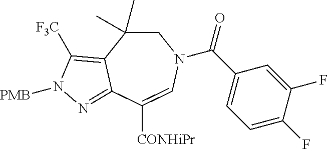

is prepared by a process comprising contacting a compound with the structure:

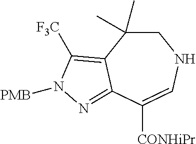

with base and 3,4-difluorobenzoyl chloride in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

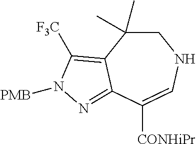

is prepared by a process comprising contacting a compound with the structure:

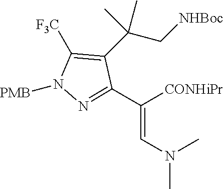

with acid in the presence of a solvent. In some embodiments, the acid is hydrochloric acid.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

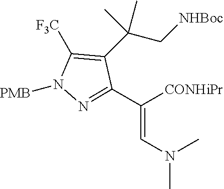

is prepared by a process comprising contacting a compound with the structure:

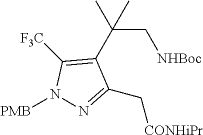

with tert-butoxy-bis(dimethylamino)methane.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

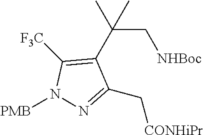

is prepared by a process comprising contacting a compound with the structure:

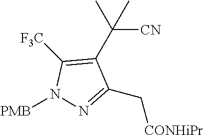

with Raney nickel, di-tert-butyl dicarbonate, and aqueous 25 weight % ammonia solution, under a hydrogen atmosphere in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

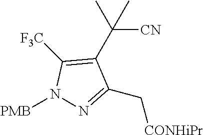

is prepared by a process comprising contacting a compound with the structure:

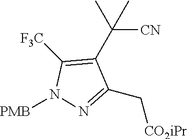

with a base followed by an amide coupling reagent and isopropylamine. In some embodiments, the base is lithium hydroxide or sodium hydroxide. In some embodiments, the amide coupling reagent is EDCI, HATU, or HOBt.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

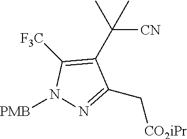

is prepared by a process comprising contacting a compound with the structure:

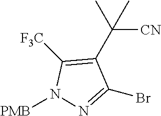

with (2-isopropoxy-2-oxoethyl)zinc(II) bromide and bromo(tri-tert-butylphosphine)palladium(I) dimer in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

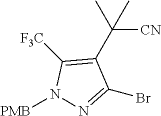

is prepared by a process comprising contacting a compound with the structure:

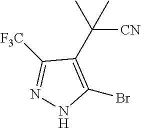

with 4-methoxybenzyl chloride and potassium carbonate in the presence of a solvent.

In some embodiments of a process for the preparation of (E)-6-(3,4-difluorobenzoyl)-N-isopropyl-4, 4-dimethyl-3-(trifluoromethyl)-1,4,5,6-tetrahydropyrazolo[3,4-d]azepine-8-carboxamide (Compound 1), the compound with the structure:

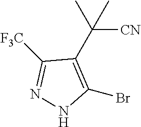

prepared by a process comprising contacting a compound with the structure:

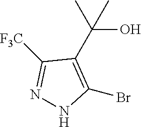

with trimethylsilyl cyanide and indium(III) bromide in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

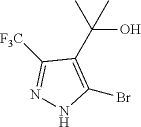

is prepared by a process comprising contacting a compound with the structure:

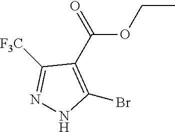

with methyl magnesium bromide in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

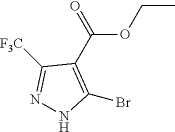

is prepared by a process comprising contacting a compound with the structure:

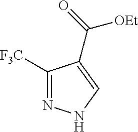 [SMILES: CCOC(=O)c1c[nH]nc1C(F)(F)F]

with bromine and sodium acetate in acetic acid.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

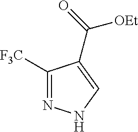 [SMILES: CCOC(=O)c1c[nH]nc1C(F)(F)F]

is prepared by a process comprising contacting a compound with the structure:

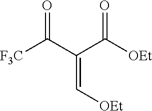 [SMILES: CCO/C=C(\C(=O)OCC)C(=O)C(F)(F)F]

with hydrazine hydrate in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

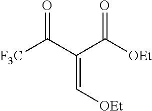 [SMILES: CCO/C=C(\C(=O)OCC)C(=O)C(F)(F)F]

is prepared by a process comprising contacting a compound with the structure:

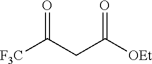 [SMILES: CCOC(=O)CC(=O)C(F)(F)F]

with triethyl orthoformate and acetic anhydride.

In another aspect is a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2):

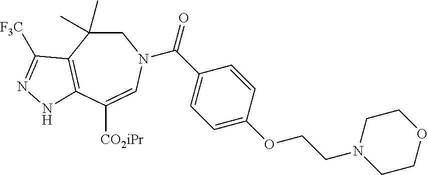

comprising contacting a compound with the structure:

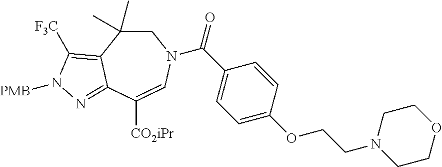

with acid followed by base. In some embodiments, the acid is trifluoroacetic acid. In some embodiments, the base is saturated aqueous sodium bicarbonate.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

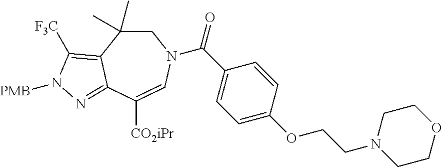

is prepared by a process comprising contacting a compound with the structure:

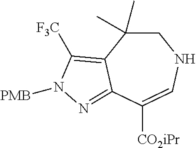

with base and 4-(2-morpholinoethoxy)benzoyl chloride hydrochloride in the presence of a solvent. In some embodiments, the base is sodium hydride, sodium bis(trimethylsilyl)amide, or lithium bis(trimethylsilyl)amide.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

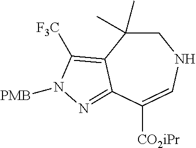

is prepared by a process comprising contacting a compound with the structure:

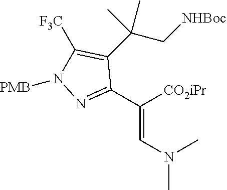

with acid in Ihe presence of a solvent. In some embodiments, the acid is

In some embodiments of a process for the preparation of (E)-isopropyl 4,4-dimethyl-6-(4-(2-morpholinoethoxy) benzoyl)-3-(trifluorometyhyl)-1,4,5,6-tetrahydropyrazolo[3,4-d]azepine-8-carboxylate (Compound 2). the compound with the structure:

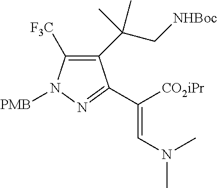

is prepared by a process comprising contacting a compuond with the structure:

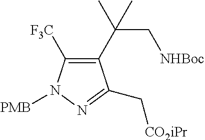

with tert-butoxy-bis(dimethylamino)methane.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

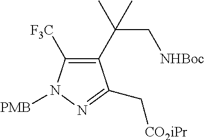

is prepared by a process comprising contacting a compound with the structure:

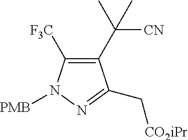

with Raney nickel, di-tert-butyl dicarbonate, and aqueous 25 weight % ammonia solution, under a hydrogen atmosphere in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

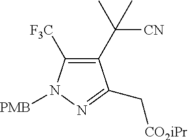

is prepared by a process comprising contacting a compound with the structure:

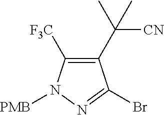

with (2-isopropoxy-2-oxoethyl)zinc(II) bromide and bromo(tri-tert-butylphosphine)palladium(I) dimer in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

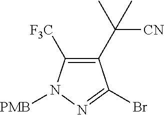

is prepared by a process comprising contacting a compound with the structure:

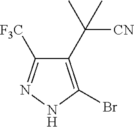

with 4-methoxybenzyl chloride and potassium carbonate in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

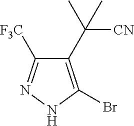

is prepared by a process comprising contacting a compound with the structure:

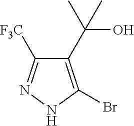

with trimethylsilyl cyanide and indium(III) bromide in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

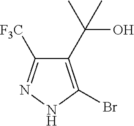

is prepared by a process comprising contacting a compound with the structure:

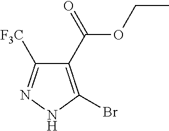

with methyl magnesium bromide in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

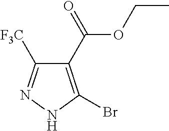

is prepared by a process comprising contacting a compound with the structure:

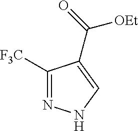 [SMILES: CCOC(=O)c1c[nH]nc1C(F)(F)F]

with bromine and sodium acetate in acetic acid.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

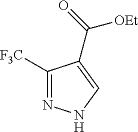 [SMILES: CCOC(=O)c1c[nH]nc1C(F)(F)F]

is prepared by a process comprising contacting a compound with the structure:

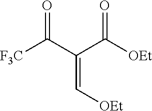 [SMILES: CCO/C=C(\C(=O)OCC)C(=O)C(F)(F)F]

with hydrazine hydrate in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

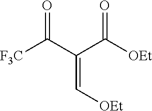 [SMILES: CCO/C=C(\C(=O)OCC)C(=O)C(F)(F)F]

is prepared by a process comprising contacting a compound with the structure:

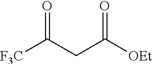 [SMILES: CCOC(=O)CC(=O)C(F)(F)F]

with triethyl orthoformate and acetic anhydride.

In another aspect is a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3):

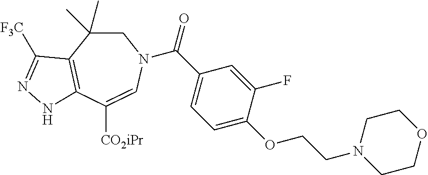

comprising contacting a compound with the structure:

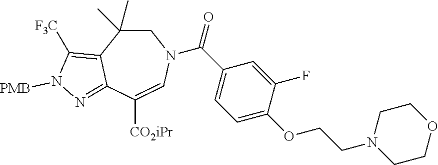

with acid followed by base. In some embodiments, the acid is trifluoroacetic acid. In some embodiments, the base is saturated aqueous sodium bicarbonate.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

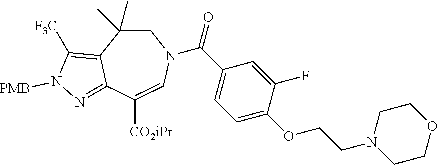

is prepared by a process comprising contacting a compound with the structure:

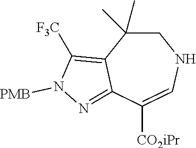

with base and 3-fluoro-4-(2-morpholinoethoxy)benzoyl chloride in the presence of a solvent. In some embodiments, the base is sodium hydride, sodium bis(trimethylsilyl)amide, or lithium bis(trimethylsilyl)amide.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

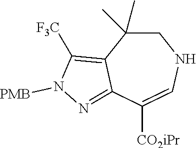

is prepared by a process comprising contacting a compound with the structure:

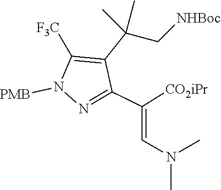

with acid in the presence of a solvent. In some embodiments, the acid is hydrochloric acid.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

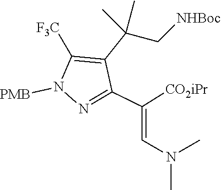

is prepared by a process comprising contacting a compound with the structure:

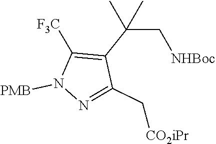

with tert-butoxy-bis(dimethylamino)methane.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

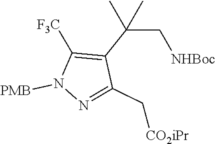

is prepared by a process comprising contacting a compound with the structure:

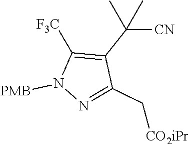

with Raney nickel, di-tert-butyl dicarbonate, and aqueous 25 weight % ammonia solution, under a hydrogen atmosphere in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

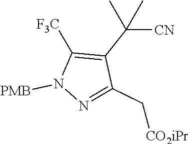

is prepared by a process comprising contacting a compound with the structure:

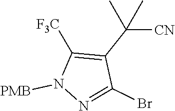

with (2-isopropoxy-2-oxoethyl)zinc(II) bromide and bromo(tri-tert-butylphosphine)palladium(I) dimer in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

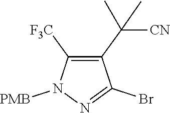

is prepared by a process comprising contacting a compound with the structure:

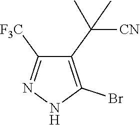

with 4-methoxybenzyl chloride and potassium carbonate in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

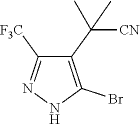

is prepared by a process comprising contacting a compound with the structure:

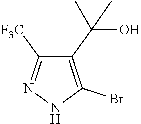

with trimethylsilyl cyanide and indium(III) bromide in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

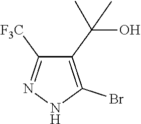

is prepared by a process comprising contacting a compound with the structure:

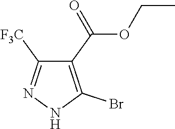

with methyl magnesium bromide in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

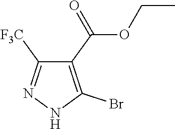

is prepared by a process comprising contacting a compound with the structure:

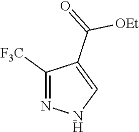 [SMILES: CCOC(=O)c1c[nH]nc1C(F)(F)F]

with bromine and sodium acetate in acetic acid.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

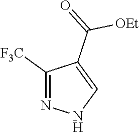 [SMILES: CCOC(=O)c1c[nH]nc1C(F)(F)F]

is prepared by a process comprising contacting a compound with the structure:

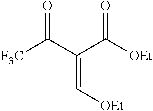 [SMILES: CCO/C=C(\C(=O)OCC)C(=O)C(F)(F)F]

with hydrazine hydrate in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

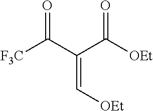 [SMILES: CCO/C=C(\C(=O)OCC)C(=O)C(F)(F)F]

is prepared by a process comprising contacting a compound with the structure:

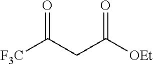 [SMILES: CCOC(=O)CC(=O)C(F)(F)F]

with triethyl orthoformate and acetic anhydride.

Further disclosed herein, is a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), comprising:

A) the reaction of a compound with the structure:

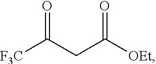 [SMILES: CCOC(=O)CC(=O)C(F)(F)F]

with triethyl orthoformate and acetic anhydride to produce a compound with the structure:

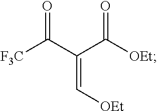 [SMILES: CCO/C=C(\C(=O)OCC)C(=O)C(F)(F)F]

B) followed by the reaction of the compound with the structure:

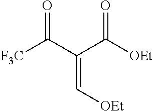 [SMILES: CCO/C=C(\C(=O)OCC)C(=O)C(F)(F)F]

with hydrazine hydrate in the presence of a solvent to produce a compound with the structure:

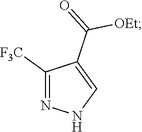 [SMILES: CCOC(=O)c1c[nH]nc1C(F)(F)F]

C) followed by the reaction of the compound with the structure:

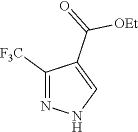 [SMILES: CCOC(=O)c1c[nH]nc1C(F)(F)F]

with bromine and sodium acetate in acetic acid to produce a compound with the structure:

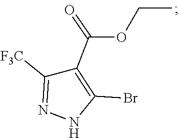

D) followed by the reaction of the compound with the structure:

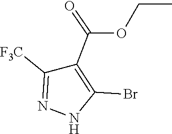

with methyl magnesium bromide in the presence of a solvent followed by treatment with an acid to produce a compound with the structure;

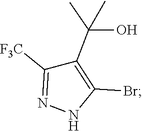

E) followed by the reaction of the compound with the structure:

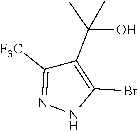

with trimethylsilyl cyanide, and indium(III) bromide in the presence of a solvent to produce a compound with the structure:

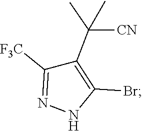

F) followed by the reaction of the compound with the structure:

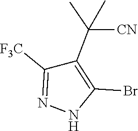

with 4-methoxybenzyl chloride and potassium carbonate in the presence of a solvent to produce a compound with the structure:

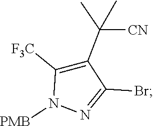

G) followed by the reaction of the compound with the structure:

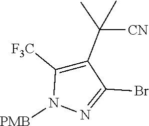

with (2-isopropoxy-2-oxoethyl)zinc(II) bromide and bromo(tri-tert-butylphosphine)palladium(I) dimer in the presence of a solvent to produce a compound with the structure:

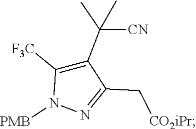

H) followed by the reaction of the compound with the structure:

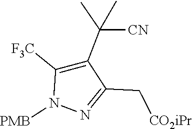

with Raney nickel, di-tert-butyl dicarbonate, aqueous 25 weight % ammonia solution, under a hydrogen atmosphere in the presence of a solvent to produce a compound with the structure:

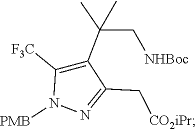

I) followed by the reaction of the compound with the structure:

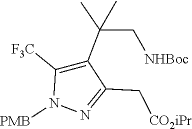

with tert-butoxy-bis(dimethylamino)methane to produce a compound with the structure:

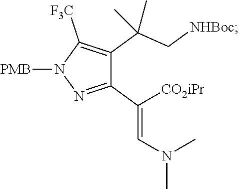

J) followed by the reaction of the compound with the structure:

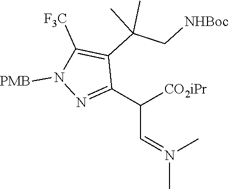

with concentrated acid in the presence of a solvent to produce a compound with the structure:

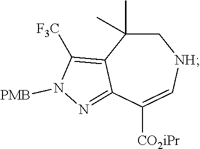

K) followed by the reaction of the compound with the structure:

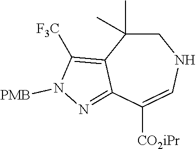

with base and 4-(2-morpholinoethoxy)benzoyl chloride hydrochloride in the presence of a solvent to produce a compound with the structure:

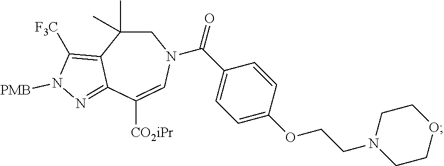

L) followed by the reaction of the compound with the structure:

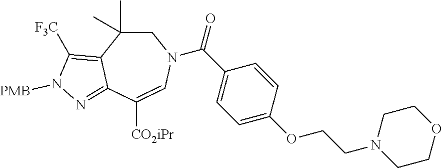

with acid to produce (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate having the structure

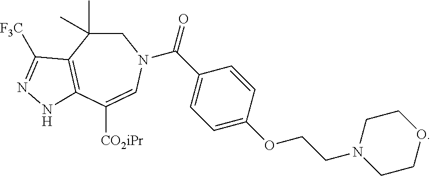

In some embodiments is a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2) further comprising treating

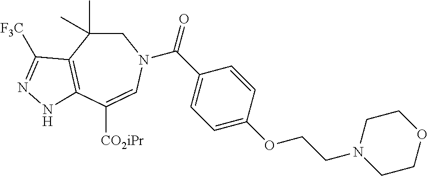

with hydrochloric acid in the presence of a solvent to produce (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate hydrochloride having the structure

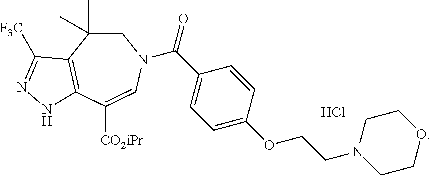

Further disclosed herein, is a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), comprising:

A) the reaction of a compound with the structure

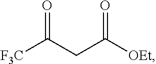 [SMILES: CCOC(=O)CC(=O)C(F)(F)F]

with triethyl orthoformate and acetic anhydride to produce a compound with the structure:

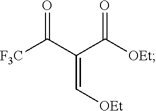 [SMILES: CCO/C=C(\C(=O)OCC)C(=O)C(F)(F)F]

B) followed by the reaction of the compound with the structure:

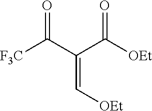 [SMILES: CCO/C=C(\C(=O)OCC)C(=O)C(F)(F)F]

with hydrazine hydrate in the presence of a solvent to produce a compound with the structure:

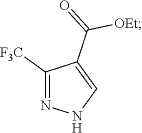 [SMILES: CCOC(=O)c1c[nH]nc1C(F)(F)F]

C) followed by the reaction of the compound with the structure:

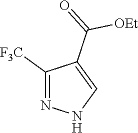 [SMILES: CCOC(=O)c1c[nH]nc1C(F)(F)F]

with bromine and sodium acetate in acetic acid to produce a compound with the structure:

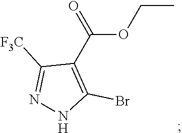

D) followed by the reaction of the compound with the structure:

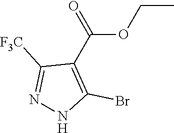

with methyl magnesium bromide in the presence of a solvent followed by treatment with an acid to produce a compound with the structure:

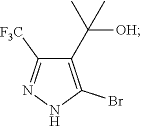

E) followed by the reaction of the compound with the structure:

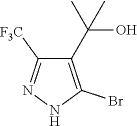

with trimethylsilyl cyanide, and indium(III) bromide in the presence of a solvent to produce a compound with the structure:

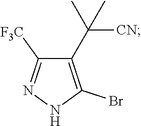

F) followed by the reaction of the compound with the structure:

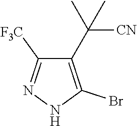

with 4-methoxybenzyl chloride and potassium carbonate in the presence of a solvent to produce a compound with the structure:

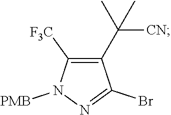

G) followed by the reaction of the compound with the structure:

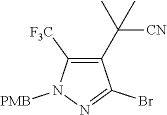

with (2-isopropoxy-2-oxoethyl)zinc(II) bromide and bromo(tri-tert-butylphosphine)palladium(I) dimer in the presence of a solvent to produce a compound with the structure:

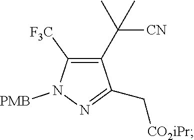

H) followed by the reaction of the compound with the structure:

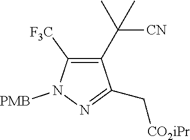

with Raney nickel, di-tert-butyl dicarbonate, aqueous 25 weight % ammonia solution, under a hydrogen atmosphere in the presence of a solvent to produce a compound with the structure:

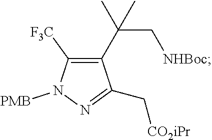

I) followed by the reaction of the compound with the structure:

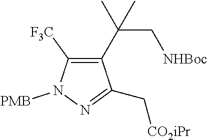

with tert-butoxy-bis(dimethylamino)methane to produce a compound with the structure:

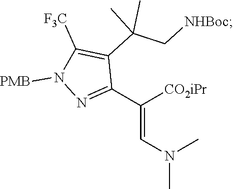

J) followed by the reaction of the compound with the structure:

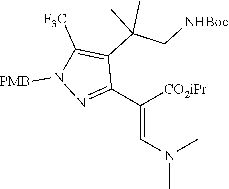

with concentrated acid in the presence of a solvent to produce a compound with the structure:

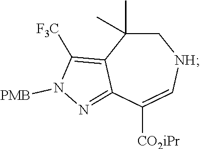

K) followed by the reaction of the compound with the structure:

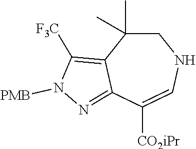

with base and 3-fluoro-4-(2-morpholinoethoxy)benzoyl chloride in the presence of a solvent to produce a compound with the structure:

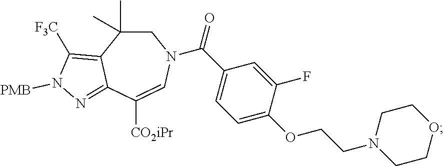

L) followed by the reaction of the compound with the structure:

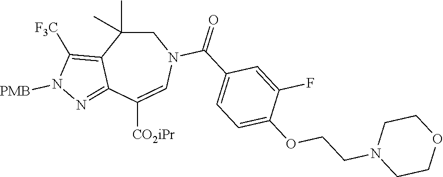

with acid to produce (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate having the structure:

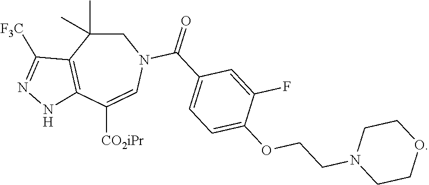

In some embodiments is a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), further comprising treating

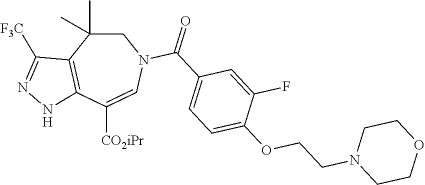

with hydrochloric acid in the presence of a solvent to produce €-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate hydrochloride having the structure:

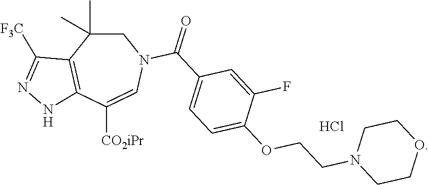

Further disclosed herein, is a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), comprising:

A) the reaction of a compound with the structure:

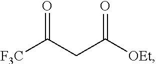 [SMILES: CCOC(=O)CC(=O)C(F)(F)F]

with triethyl orthoformate and acetic anhydride to produce a compound with the structure:

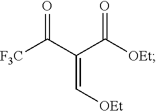 [SMILES: CCO/C=C(\C(=O)OCC)C(=O)C(F)(F)F]

B) followed by the reaction of the compound with the structure:

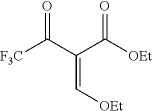 [SMILES: CCO/C=C(\C(=O)OCC)C(=O)C(F)(F)F]

with hydrazine hydrate in the presence of a solvent to produce a compound with the structure:

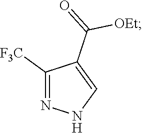 [SMILES: CCOC(=O)c1c[nH]nc1C(F)(F)F]

C) followed by the reaction of the compound with the structure:

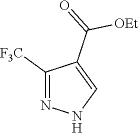 [SMILES: CCOC(=O)c1c[nH]nc1C(F)(F)F]

with bromine and sodium acetate in acetic acid to produce a compound with the structure:

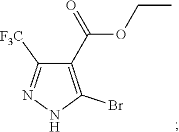

D) followed by the reaction of the compound with the structure:

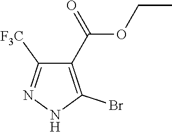

with methyl magnesium bromide in the presence of a solvent followed by treatment with an acid to produce a compound with the structure:

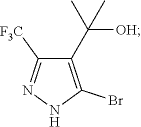

E) followed by the reaction of the compound with the structure:

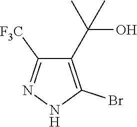

with trimethylsilyl cyanide, and indium(III) bromide in the presence of a solvent to produce a compound with the structure:

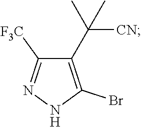

F) followed by the reaction of the compound with the structure:

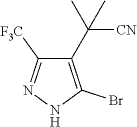

with 4-methoxybenzyl chloride and potassium carbonate in the presence of a solvent to produce a compound with the structure:

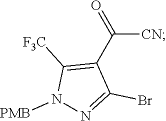

G) followed by the reaction of the compound with the structure:

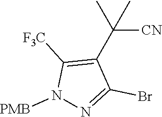

with (2-isopropoxy-2-oxoethyl)zinc(II) bromide and bromo(tri-tert-butylphosphine)palladium(I) dimer in the presence of a solvent to produce a compound with the structure:

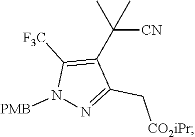

H) Followed by the reaction of a compound with the structure:

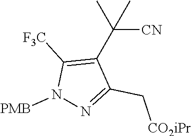

with lithium hydroxide followed by an amide coupling agent and isopropylamine to produce a compound with the structure:

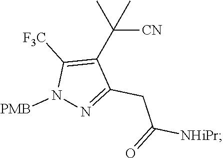

I) followed by the reaction of the compound with the structure:

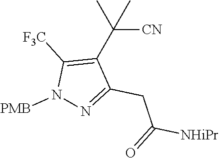

with Raney nickel, di-tert-butyl dicarbonate, aqueous 25 weight % ammonia solution, under a hydrogen atmosphere in the presence of a solvent to produce a compound with the structure:

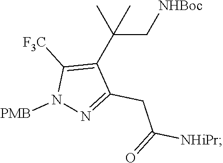

J) followed by the reaction of the compound with the structure:

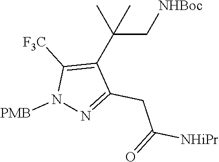

with tert-butoxy-bis(dimethylamino)methane to produce a compound with the structure:

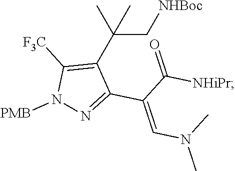

K) followed by the reaction of the compound with the structure:

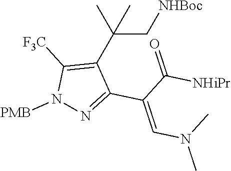

with concentrated acid in the presence of a solvent to produce a compound with the structure:

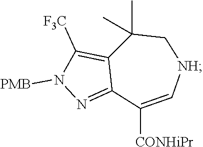

L) followed by the reaction of the compound with the structure

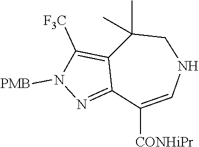

with base and 3,4-difluorobenzoyl chloride in the presence of a solvent to produce a compound with the structure:

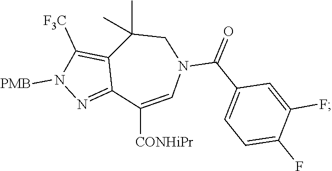

M) followed by the reaction of the compound with the structure:

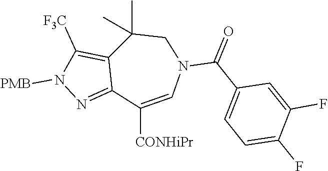

with acid to produce 186 -6-(3,4-difluorobenzoyl)-N-isopropyl-4,4-dimethyl-3-(trifluoromethyl)-1,4,5,6-tetrahydropyrazolo[3,4-d]azepine-8-carboxamide having the structure:

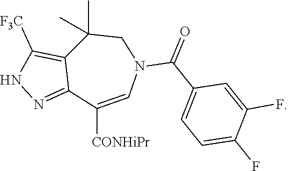

Further disclosed herein is a compound having the structure:

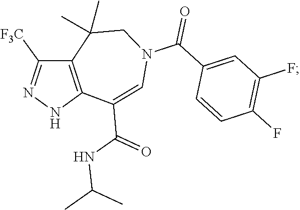 [SMILES: CC(C)NC(=O)C1=CN(C(=O)c2ccc(F)c(F)c2)CC(C)(C)c2c(C(F)(F)F)n[nH]c21]

or a pharmaceutically acceptable salt thereof.

Further disclosed herein is a compound having the structure:

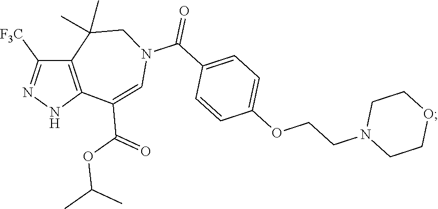 [SMILES: CC(C)OC(=O)C1=CN(C(=O)c2ccc(OCCN3CCOCC3)cc2)CC(C)(C)c2c(C(F)(F)F)n[nH]c21]

or a pharmaceutically acceptable salt thereof.

Further disclosed herein is a compound having the structure:

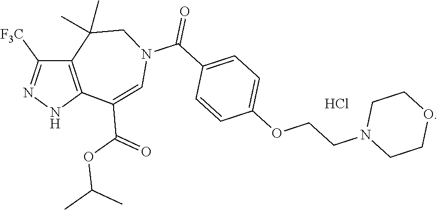 [SMILES: CC(C)OC(=O)C1=CN(C(=O)c2ccc(OCCN3CCOCC3)cc2)CC(C)(C)c2c(C(F)(F)F)n[nH]c21 ; Cl]

Further disclosed herein is a compound having the structure:

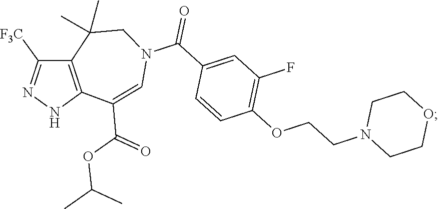 [SMILES: CC(C)OC(=O)C1=CN(C(=O)c2ccc(OCCN3CCOCC3)c(F)c2)CC(C)(C)c2c(C(F)(F)F)n[nH]c21]

or a pharmaceutically acceptable salt thereof.

Further disclosed herein is a compound having the structure:

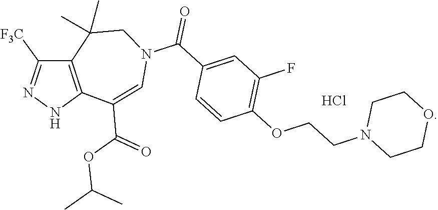 [SMILES: CC(C)OC(=O)C1=CN(C(=O)c2ccc(OCCN3CCOCC3)c(F)c2)CC(C)(C)c2c(C(F)(F)F)n[nH]c21 ; Cl]

In some embodiments of the processes described herein, the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof.

In some embodiments of the processes described herein, the FXR modulator is (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof.

In some embodiments of the processes described herein, the FXR modulator is (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Good manufacturing practices are usually required for large scale manufacture of clinically useful drug candidates. Provided herein are certain processes and methods for the manufacture of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

### Definitions

As used in the specification and appended claims, unless specified to the contrary, the following terms have the meaning indicated below.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range.

The term “comprising” (and related terms such as “comprise” or “comprises” or “having” or “including”) is not intended to exclude that which in other certain embodiments, for example, an embodiment of any composition of matter, composition, method, or process, or the like, described herein, “consist of” or “consist essentially of” the described features.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has been made.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “pharmaceutical combination” as used herein, means a product that results from the mixing or combining of more than one active ingredient and includes both fixed and non-fixed combinations of the active ingredients. The term “fixed combination” means that the active ingredients, are both administered to a patient simultaneously in the form of a single entity or dosage.

The term “non-fixed combination” means that the active ingredients, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “co-administration” or the like, as used herein, are meant to encompass administration of the selected therapeutic agents to a single patient, and are intended to include treatment regimens in which the agents are administered by the same or different route of administration or at the same or different time.

The term “activator” is used in this specification to denote any molecular species that results in activation of the indicated receptor, regardless of whether the species itself binds to the receptor or a metabolite of the species binds to the receptor when the species is administered topically. Thus, the activator can be a ligand of the receptor or it can be an activator that is metabolized to the ligand of the receptor, i.e., a metabolite that is formed in tissue and is the actual ligand.

The term “antagonist” as used herein, refers to a small-molecule agent that binds to a nuclear hormone receptor and subsequently decreases the agonist induced transcriptional activity of the nuclear hormone receptor.

The term “agonist” as used herein, refers to a small-molecule agent that binds to a nuclear hormone receptor and subsequently increases nuclear hormone receptor transcriptional activity in the absence of a known agonist.

The term “inverse agonist” as used herein, refers to a small-molecule agent that binds to a nuclear hormone receptor and subsequently decreases the basal level of nuclear hormone receptor transcriptional activity that is present in the absence of a known agonist.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target.

### Compounds

Described herein are FXR modulatators, and pharmaceutical compositions that include such FXR modulatators, for use in the treatment of diseases, disorders or conditions that would benefit from FXR modulation. In some embodiments is the administration of an FXR modulator described herein to a mammal in the treatment of diseases, disorders or conditions that would benefit from FXR modulation, wherein the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

In some embodiments is the administration of an FXR modulator described herein to a mammal in the treatment of diseases, disorders or conditions that would benefit from FXR modulation, wherein the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. Compound 1 has the structure:

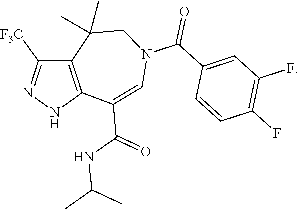 [SMILES: CC(C)NC(=O)C1=CN(C(=O)c2ccc(F)c(F)c2)CC(C)(C)c2c(C(F)(F)F)n[nH]c21]

In some embodiments is the administration of an FXR modulator described herein to a mammal in the treatment of diseases, disorders or conditions that would benefit from FXR modulation, wherein the FXR modulator is (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. Compound 2 has the structure:

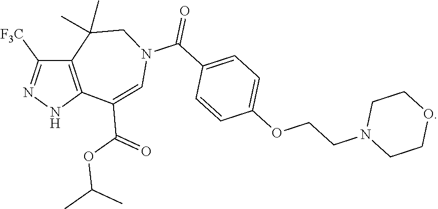 [SMILES: CC(C)OC(=O)C1=CN(C(=O)c2ccc(OCCN3CCOCC3)cc2)CC(C)(C)c2c(C(F)(F)F)n[nH]c21]

In some embodiments is the administration of an FXR modulator described herein to a mammal in the treatment of diseases, disorders or conditions that would benefit from FXR modulation, wherein the FXR modulator is (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. Compound 3 has the structure:

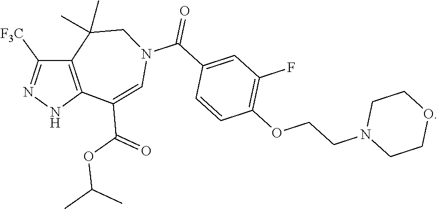 [SMILES: CC(C)OC(=O)C1=CN(C(=O)c2ccc(OCCN3CCOCC3)c(F)c2)CC(C)(C)c2c(C(F)(F)F)n[nH]c21]

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

In some embodiments, the pharmaceutically acceptable salt of Compound 1 is an acetate, benzoate, besylate, bitartrate, carbonate, citrate, fumarate, gluconate, hydrobromide, hydrochloride, maleate, mesylate, nitrate, phosphate, salicylate, succinate, sulfate, or tartrate salt. In some embodiments, the pharmaceutically acceptable salt of Compound 1 is a mono-hydrochloride salt. In further embodiments, the pharmaceutically acceptable salt of Compound 1 is a mono-hydrochloride salt.

In some embodiments, the pharmaceutically acceptable salt of Compound 2 is an acetate, benzoate, besylate, bitartrate, carbonate, citrate, fumarate, gluconate, hydrobromide, hydrochloride, maleate, mesylate, nitrate, phosphate, salicylate, succinate, sulfate, or tartrate salt. In some embodiments, the pharmaceutically acceptable salt of Compound 2 is a mono-hydrochloride salt. In further embodiments, the pharmaceutically acceptable salt of Compound 2 is a mono-hydrochloride salt.

In some embodiments, the pharmaceutically acceptable salt of Compound 3 is an acetate, benzoate, besylate, bitartrate, carbonate, citrate, fumarate, gluconate, hydrobromide, hydrochloride, maleate, mesylate, nitrate, phosphate, salicylate, succinate, sulfate, or tartrate salt. In some embodiments, the pharmaceutically acceptable salt of Compound 3 is a mono-hydrochloride salt. In further embodiments, the pharmaceutically acceptable salt of Compound 3 is a mono-hydrochloride salt.

### Tautomers

In some situations, Compound 1, Compound 2, or Compound 3 may exist as tautomers. All tautomers are included within the scope of the compounds presented herein.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. Increased levels of deuterium incorporation produce a detectable kinetic isotope effect (KIE) that may affect the pharmacokinetic, pharmacologic and/or toxicologic parameters of Compound 1, Compound 2, or Compound 3 in comparison to Compound 1, Compound 2, or Compound 3 having naturally occurring levels of deuterium. In some embodiments, the isotopically labeled compound, or a pharmaceutically acceptable salt thereof, is prepared by any suitable method.

In some embodiments, at least one hydrogen in Compound 1 is replaced with deuterium. In some embodiments of the methods described herein, at least one hydrogen in Compound 1 is replaced with deuterium. In some embodiments of the pharmaceutical compositions described herein, at least one hydrogen in Compound 1 is replaced with deuterium.

In some embodiments, at least one hydrogen in Compound 2 is replaced with deuterium. In some embodiments of the methods described herein, at least one hydrogen in Compound 2 is replaced with deuterium. In some embodiments of the pharmaceutical compositions described herein, at least one hydrogen in Compound 2 is replaced with deuterium.

In some embodiments, at least one hydrogen in Compound 3 is replaced with deuterium. In some embodiments of the methods described herein, at least one hydrogen in Compound 3 is replaced with deuterium. In some embodiments of the pharmaceutical compositions described herein, at least one hydrogen in Compound 3 is replaced with deuterium.

In some embodiments, the compounds described herein are labeled by other means, including, but not limited to, the use of chromophores or fluorescent moieties, bioluminescent labels, or chemiluminescent labels.

### Process for Preparation

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein.

In some embodiments is a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1):

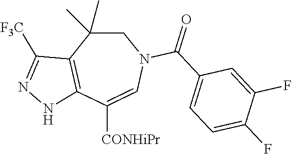

comprising contacting a compound with the structure:

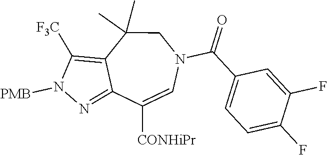

with acid followed by base. In some embodiments, the acid is trifluoroacetic acid. In some embodiments, the base is saturated aqueous sodium bicarbonate.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

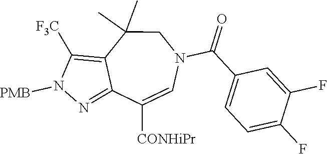

is prepared by a process comprising contacting a compound with the structure:

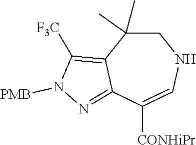

with base and 3,4-difluorobenzoyl chloride in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

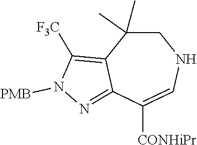

is prepared by a process comprising contacting a compound with the structure:

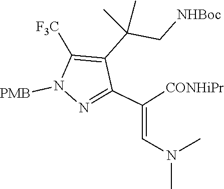

with acid in the presence of a solvent. In some embodiments, the acid is hydrochloric acid.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

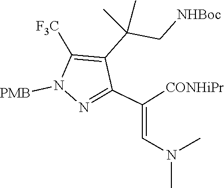

is prepared by a process comprising contacting a compound with the structure:

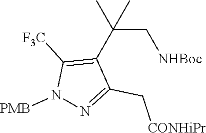

with tert-butoxy-bis(dimethylamino)methane.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

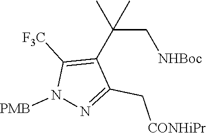

is prepared by a process comprising contacting a compound with the structure:

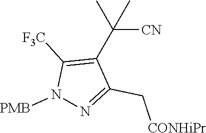

with Raney nickel, di-tert-butyl dicarbonate, and aqueous 25 weight % ammonia solution, under a hydrogen atmosphere in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

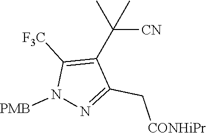

is prepared by a process comprising contacting a compound with the structure:

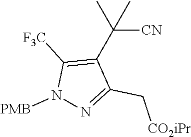

with a base followed by an amide coupling reagent and isopropylamine. In some embodiments, the base is lithium hydroxide or sodium hydroxide. In some embodiments, the amide coupling reagent is EDCI, HATU, or HOBt.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

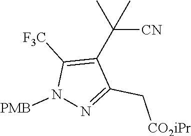

is prepared by a process comprising contacting a compound with the structure:

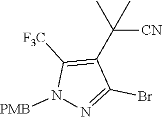

with (2-isopropoxy-2-oxoethyl)zinc(II) bromide and bromo(tri-tert-butylphosphine) palladium(I) dimer in the presence of a solvent.

In some embodiments of a process for the preparation of (E)-6-(3,4-difluorobenzoyl)-N-isopropyl-4, 4-dimethyl-3-(trifluoromelhyl)-1,4,5.6-tetrahydropyrazolo[3.4-d]azepine-8-carboxamide (Compound 1), the compound with the structure:

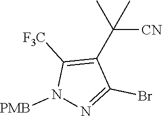

is prepared by a process comprising contascting a compound with the structure:

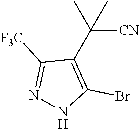

with 4-methoxybenzyl chloride and potassium carbonate in the presence of a solvent.

In some embodiments of a process for the preparation of (E)-6-(3,4-difluorobenzoyl)-N-isopropyl-4, 4-dimcthyl-3-(trifluoromethyl)-1,4,5,6-tetrahydropyrazolo[3,4-d]azepinc-8-carboxamide (Compound 1), the compound with the structure:

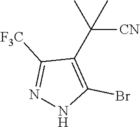

prepared by a process comprising contacting a compound with the structure:

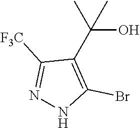

with trimethylsilyl cyanide and indium(III) bromide in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

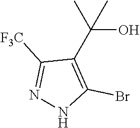

is prepared by a process comprising contacting a compound with the structure:

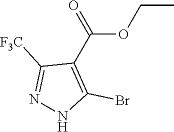

with methyl magnesium bromide in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

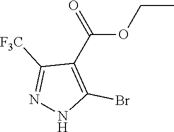

is prepared by a process comprising contacting a compound with the structure:

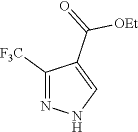 [SMILES: CCOC(=O)c1c[nH]nc1C(F)(F)F]

with bromine and sodium acetate in acetic acid.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

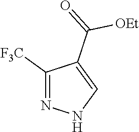 [SMILES: CCOC(=O)c1c[nH]nc1C(F)(F)F]

is prepared by a process comprising contacting a compound with the structure:

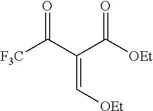 [SMILES: CCO/C=C(\C(=O)OCC)C(=O)C(F)(F)F]

with hydrazine hydrate in the presence of a solvent.

In some embodiments of a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), the compound with the structure:

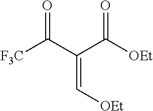 [SMILES: CCO/C=C(\C(=O)OCC)C(=O)C(F)(F)F]

is prepared by a process comprising contacting a compound with the structure:

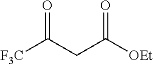 [SMILES: CCOC(=O)CC(=O)C(F)(F)F]

with triethyl orthoformate and acetic anhydride.

In some embodiments is a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2):

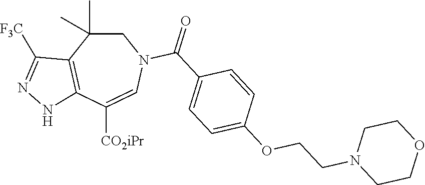

comprising contacting a compound with the structure:

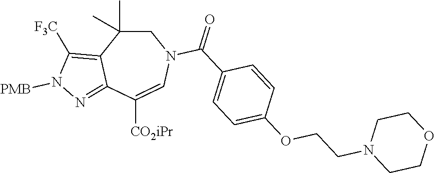

with acid followed by base. In some embodiments, the acid is trifluoroacetic acid. In some embodiments, the base is saturated aqueous sodium bicarbonate.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

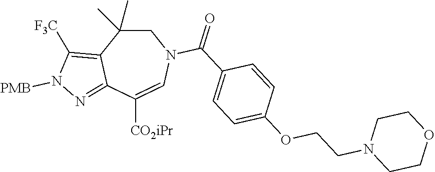

is prepared by a process comprising contacting a compound with the structure:

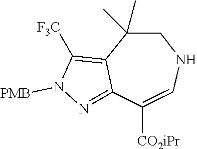

with base and 4-(2-morpholinoethoxy)benzoyl chloride hydrochloride in the presence of a solvent. In some embodiments, the base is sodium hydride, sodium bis(trimethylsilyl)amide, or lithium bis(trimethylsilyl)amide.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

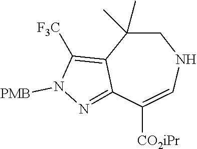

is prepared by a process comprising contacting a compound with the structure:

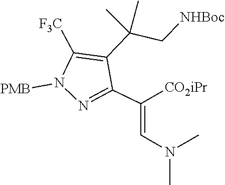

with acid in the presence of a solvent. In some embodiments, the acid is hydrochloric acid.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

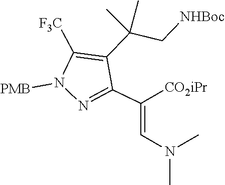

is prepared by a process comprising contacting a compound with the structure:

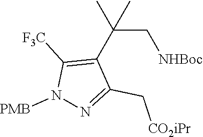

with tert-butoxy-bis(dimethylamino)methane.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

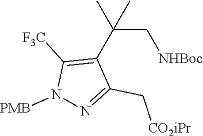

is prepared by a process comprising contacting a compound with the structure:

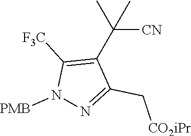

with Raney nickel, di-tert-butyl dicarbonate, and aqueous 25 weight % ammonia solution, under a hydrogen atmosphere in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

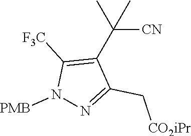

is prepared by a process comprising contacting a compound with the structure:

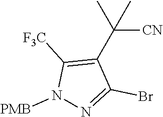

with (2-isopropoxy-2-oxoethyl)zinc(II) bromide and bromo(tri-tert-butylphosphine)palladium(I) dimer in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

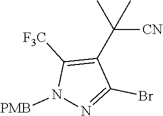

is prepared by a process comprising contacting a compound with the structure:

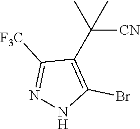

with 4-methoxybenzyl chloride and potassium carbonate in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

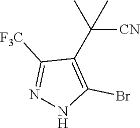

is prepared by a process comprising contacting a compound with the structure:

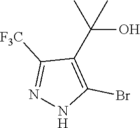

with trimethylsilyl cyanide and indium(III) bromide in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

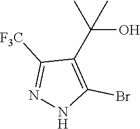

is prepared by a process comprising contacting a compound with the structure:

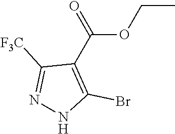

with methyl magnesium bromide in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

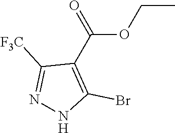

is prepared by a process comprising contacting a compound with the structure:

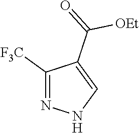 [SMILES: CCOC(=O)c1c[nH]nc1C(F)(F)F]

with bromine and sodium acetate in acetic acid.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

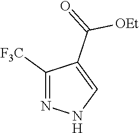 [SMILES: CCOC(=O)c1c[nH]nc1C(F)(F)F]

is prepared by a process comprising contacting a compound with the structure:

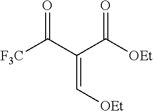 [SMILES: CCO/C=C(\C(=O)OCC)C(=O)C(F)(F)F]

with hydrazine hydrate in the presence of a solvent.

In some embodiments of a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), the compound with the structure:

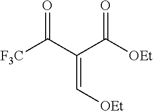 [SMILES: CCO/C=C(\C(=O)OCC)C(=O)C(F)(F)F]

is prepared by a process comprising contacting a compound with the structure:

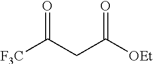 [SMILES: CCOC(=O)CC(=O)C(F)(F)F]

with triethyl orthoformate and acetic anhydride.

In some embodiments is a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3):

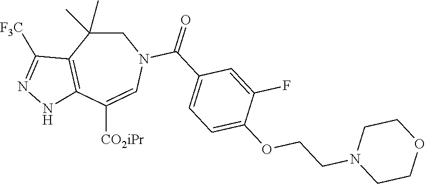

comprising contacting a compound with the structure:

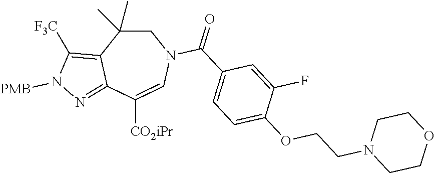

with acid followed by base. In some embodiments, the acid is trifluoroacetic acid. In some embodiments, the base is saturated aqueous sodium bicarbonate.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

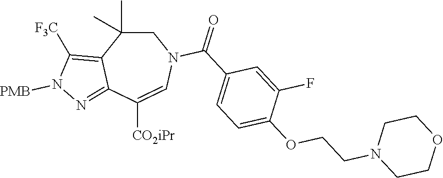

is prepared by a process comprising contacting a compound with the structure:

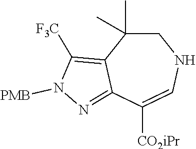

with base and 3-fluoro-4-(2-morpholinoethoxy)benzoyl chloride in the presence of a solvent. In some embodiments, the base is sodium hydride, sodium bis(trimethylsilyl)amide, or lithium bis(trimethylsilyl)amide.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

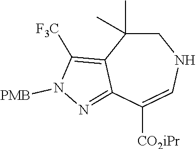

is prepared by a process comprising contacting a compound with the structure:

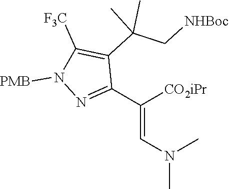

with acid in the presence of a solvent. In some embodiments, the acid is hydrochloric acid.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

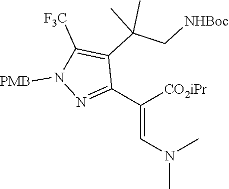

is prepared by a process comprising contacting a compound with the structure:

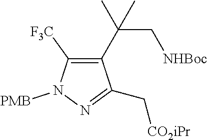

with tert-butoxy-bis(dimethylamino)methane.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

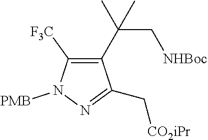

is prepared by a process comprising contacting a compound with the structure:

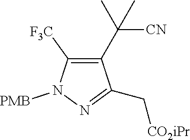

with Raney nickel, di-tert-butyl dicarbonate, and aqueous 25 weight % ammonia solution, under a hydrogen atmosphere in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

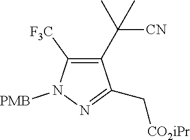

is prepared by a process comprising contacting a compound with the structure:

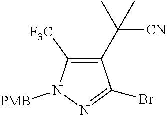

with (2-isopropoxy-2-oxoethyl)zinc(II) bromide and bromo(tri-tert-butylphosphine)palladium(I) dimer in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

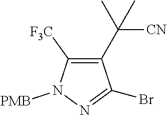

is prepared by a process comprising contacting a compound with the structure:

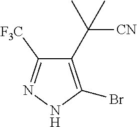

with 4-methoxybenzyl chloride and potassium carbonate in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

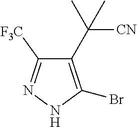

is prepared by a process comprising contacting a compound with the structure:

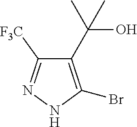

with trimethylsilyl cyanide and indium(III) bromide in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

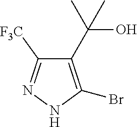

is prepared by a process comprising contacting a compound with the structure:

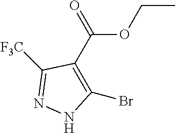

with methyl magnesium bromide in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

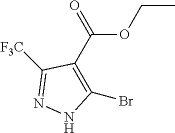

is prepared by a process comprising contacting a compound with the structure:

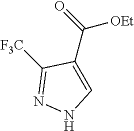 [SMILES: CCOC(=O)c1c[nH]nc1C(F)(F)F]

with bromine and sodium acetate in acetic acid.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

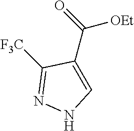 [SMILES: CCOC(=O)c1c[nH]nc1C(F)(F)F]

is prepared by a process comprising contacting a compound with the structure:

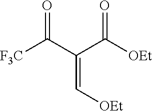 [SMILES: CCO/C=C(\C(=O)OCC)C(=O)C(F)(F)F]

with hydrazine hydrate in the presence of a solvent.

In some embodiments of a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), the compound with the structure:

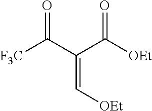 [SMILES: CCO/C=C(\C(=O)OCC)C(=O)C(F)(F)F]

is prepared by a process comprising contacting a compound with the structure:

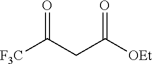 [SMILES: CCOC(=O)CC(=O)C(F)(F)F]

with triethyl orthoformate and acetic anhydride.

Further disclosed herein, is a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), comprising:

A) the reaction of a compound with the structure:

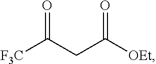 [SMILES: CCOC(=O)CC(=O)C(F)(F)F]

with triethyl orthoformate and acetic anhydride to produce a compound with the structure:

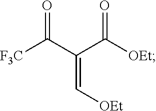 [SMILES: CCO/C=C(\C(=O)OCC)C(=O)C(F)(F)F]

B) followed by the reaction of the compound with the structure:

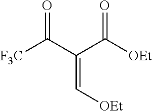 [SMILES: CCO/C=C(\C(=O)OCC)C(=O)C(F)(F)F]

with hydrazine hydrate in the presence of a solvent to produce a compound with the structure:

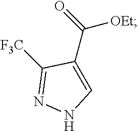 [SMILES: CCOC(=O)c1c[nH]nc1C(F)(F)F]

C) followed by the reaction of the compound with the structure:

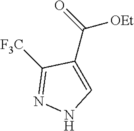 [SMILES: CCOC(=O)c1c[nH]nc1C(F)(F)F]

with bromine and sodium acetate in acetic acid to produce a compound with the structure:

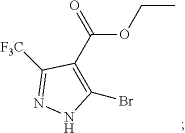

D) followed by the reaction of the compound with the structure:

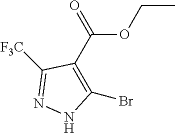

with methyl magnesium bromide in the presence of a solvent followed by treatment with an acid to produce a compound with the structure;

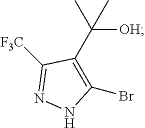

E) followed by the reaction of the compound with the structure:

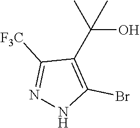

with trimethylsilyl cyanide, and indium(III) bromide in the presence of a solvent to produce a compound with the structure:

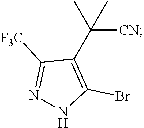

F) followed by the reaction of the compound with the structure:

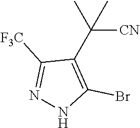

with 4-methoxybenzyl chloride and potassium carbonate in the presence of a solvent to produce a compound with the structure:

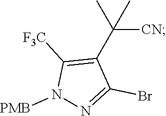

G) followed by the reaction of the compound with the structure:

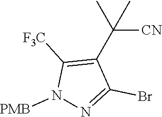

with (2-isopropoxy-2-oxoethyl)zinc(II) bromide and bromo(tri-tert-butylphosphine)palladium(I) dimer in the presence of a solvent to produce a compound with the structure:

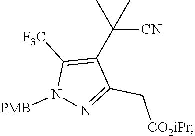

H) followed by the reaction of the compound with the structure:

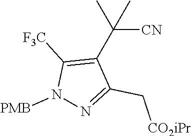

with Raney nickel, di-tert-butyl dicarbonate, aqueous 25 weight % ammonia solution, under a hydrogen atmosphere in the presence of a solvent to produce a compound with the structure:

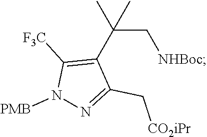

I) followed by the reaction of the compound with the structure:

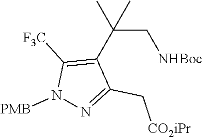

with tert-butoxy-bis(dimethylamino)methane to produce a compound with the structure:

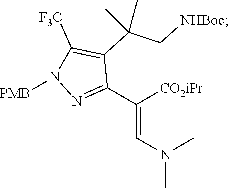

J) followed by the reaction of the compound with the structure:

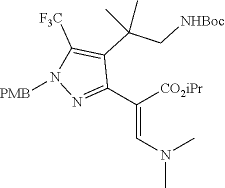

with concentrated acid in the presence of a solvent to produce a compound with the structure:

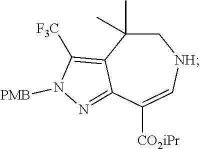

K) followed by the reaction of the compound with the structure:

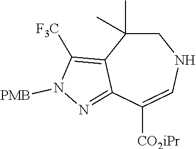

with base and 4-(2-morpholinoethoxy)benzoyl chloride hydrochloride in the presence of a solvent to produce a compound with the structure:

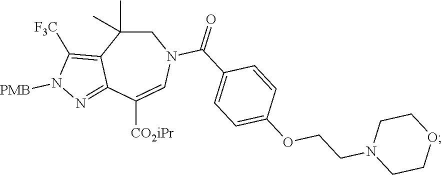

L) followed by the reaction of the compound with the structure:

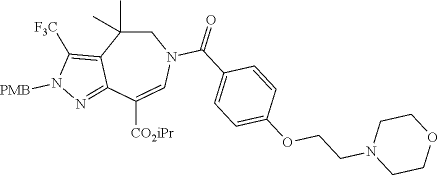

with acid to produce (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate having the structure

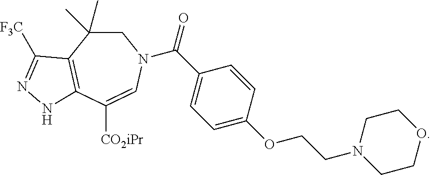

In some embodiments is a process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2) further comprising treating

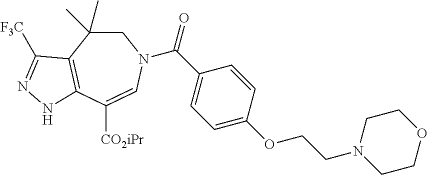

with hydrochloric acid in the presence of a solvent to produce (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate hydrochloride having the structure

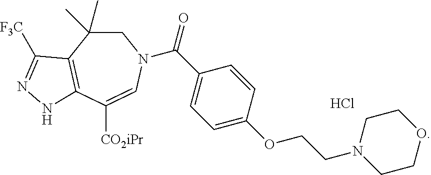

Further disclosed herein, is a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), comprising:

A) the reaction of a compound with the structure

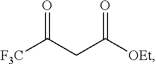 [SMILES: CCOC(=O)CC(=O)C(F)(F)F]

with triethyl orthoformate and acetic anhydride to produce a compound with the structure:

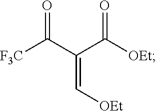 [SMILES: CCO/C=C(\C(=O)OCC)C(=O)C(F)(F)F]

B) followed by the reaction of the compound with the structure:

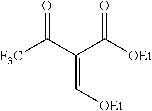 [SMILES: CCO/C=C(\C(=O)OCC)C(=O)C(F)(F)F]

with hydrazine hydrate in the presence of a solvent to produce a compound with the structure:

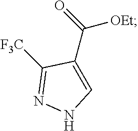 [SMILES: CCOC(=O)c1c[nH]nc1C(F)(F)F]

C) followed by the reaction of the compound with the structure:

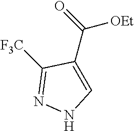 [SMILES: CCOC(=O)c1c[nH]nc1C(F)(F)F]

with bromine and sodium acetate in acetic acid to produce a compound with the structure:

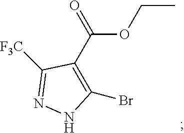

D) followed by the reaction of the compound with the structure:

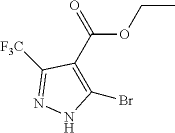

with methyl magnesium bromide in the presence of a solvent followed by treatment with an acid to produce a compound with the structure:

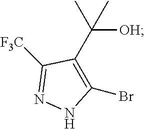

E) followed by the reaction of the compound with the structure:

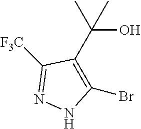

with trimethylsilyl cyanide, and indium(III) bromide in the presence of a solvent to produce a compound with the structure:

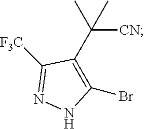

F) followed by the reaction of the compound with the structure:

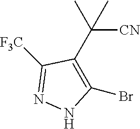

with 4-methoxybenzyl chloride and potassium carbonate in the presence of a solvent to produce a compound with the structure:

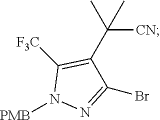

G) followed by the reaction of the compound with the structure:

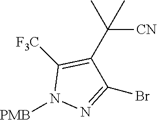

with (2-isopropoxy-2-oxoethyl)zinc(II) bromide and bromo(tri-tert-butylphosphine)palladium(I) dimer in the presence of a solvent to produce a compound with the structure:

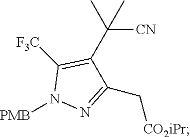

H) followed by the reaction of the compound with the structure:

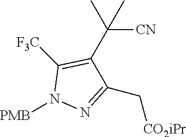

with Raney nickel, di-tert-butyl dicarbonate, aqueous 25 weight % ammonia solution, under a hydrogen atmosphere in the presence of a solvent to produce a compound with the structure:

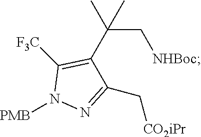

I) followed by the reaction of the compound with the structure:

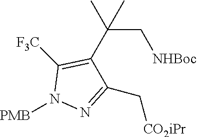

with tert-butoxy-bis(dimethylamino)methane to produce a compound with the structure:

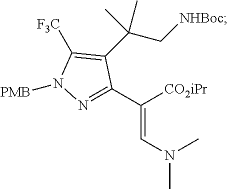

J) followed by the reaction of the compound with the structure:

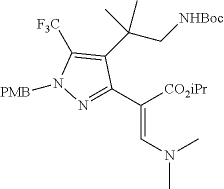

with concentrated acid in the presence of a solvent to produce a compound with the structure:

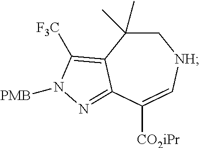

K) followed by the reaction of the compound with the structure:

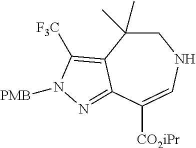

with base and 3-fluoro-4-(2-morpholinoethoxy)benzoyl chloride in the presence of a solvent to produce a compound with the structure:

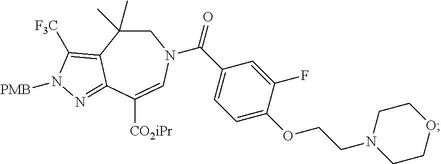

L) followed by the reaction of the compound with the structure:

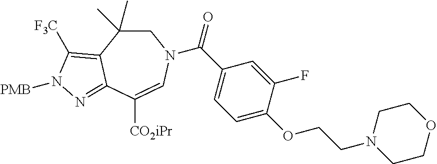

with acid to produce (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate having the structure:

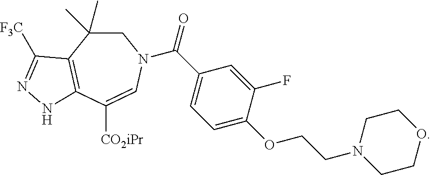

In some embodiments is a process for the preparation of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), further comprising treating

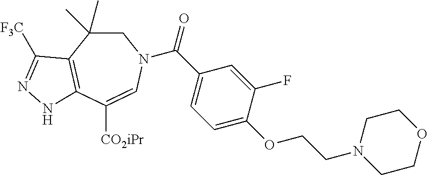

with hydrochloric acid in the presence of a solvent to produce (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate hydrochloride having the structure:

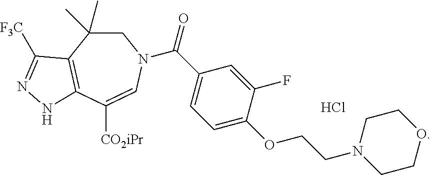

Further disclosed herein, is a process for the preparation of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), comprising:

A) the reaction of a compound with the structure:

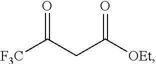 [SMILES: CCOC(=O)CC(=O)C(F)(F)F]

with triethyl orthoformate and acetic anhydride to produce a compound with the structure:

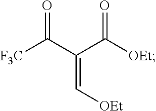 [SMILES: CCO/C=C(\C(=O)OCC)C(=O)C(F)(F)F]

B) followed by the reaction of the compound with the structure:

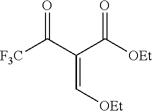 [SMILES: CCO/C=C(\C(=O)OCC)C(=O)C(F)(F)F]

with hydrazine hydrate in the presence of a solvent to produce a compound with the structure:

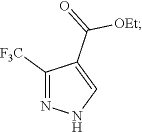 [SMILES: CCOC(=O)c1c[nH]nc1C(F)(F)F]

C) followed by the reaction of the compound with the structure:

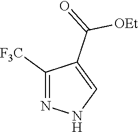 [SMILES: CCOC(=O)c1c[nH]nc1C(F)(F)F]

with bromine and sodium acetate in acetic acid to produce a compound with the structure:

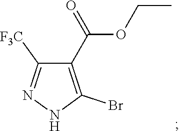

D) followed by the reaction of the compound with the structure:

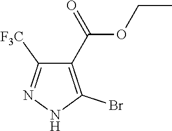

with methyl magnesium bromide in the presence of a solvent followed by treatment with an acid to produce a compound with the structure:

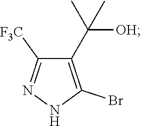

E) followed by the reaction of the compound with the structure:

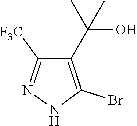

with trimethylsilyl cyanide, and indium(III) bromide in the presence of a solvent to produce a compound with the structure:

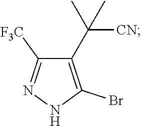

F) followed by the reaction of the compound with the structure:

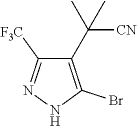

with 4-methoxybenzyl chloride and potassium carbonate in the presence of a solvent to produce a compound with the structure:

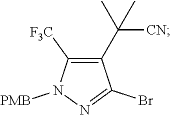

G) followed by the reaction of the compound with the structure:

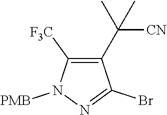

with (2-isopropoxy-2-oxoethyl)zinc(II) bromide and bromo(tri-tert-butylphosphine)palladium(I) dimer in the presence of a solvent to produce a compound with the structure:

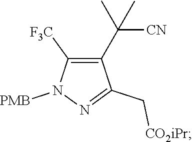

H) Followed by the reaction of a compound with the structure:

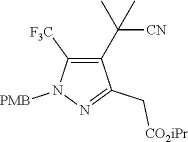

with lithium hydroxide followed by an amide coupling agent and isopropylamine to produce a compound with the structure:

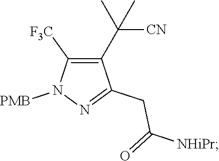

I) followed by the reaction of the compound with the structure:

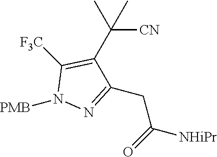

with Raney nickel, di-tert-butyl dicarbonate, aqueous 25 weight % ammonia solution, under a hydrogen atmosphere in the presence of a solvent to produce a compound with the structure:

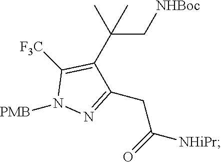

J) followed by the reaction of the compound with the structure:

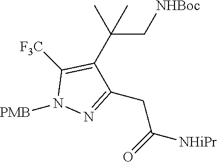

with tert-butoxy-bis(dimethylamino)methane to produce a compound with the structure:

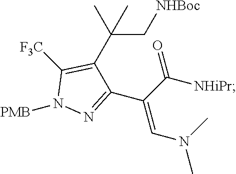

K) followed by the reaction of the compound with the structure:

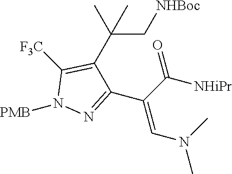

With concentrated acid in the presence of a solvent to produce a compound with the structure:

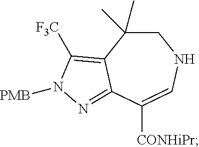

L) followed by the reaction of the compound with the structure

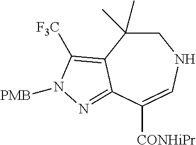

with base and 3,4-difluorobenzoyl chloride in the presence of a solvent to produce a compound with the structure:

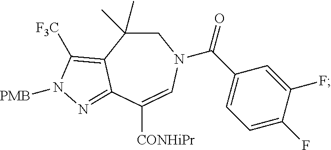

M) followed by the reaction of the compound with the structure:

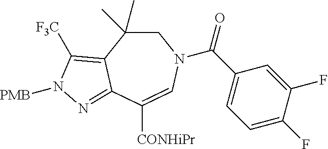

with acid to produce €-6-(3,4-difluorobenzoyl)-N-isopropyl-4,4-dimethyl-3-(trifluoromethyl)-1,4,5,6-tetrahydropyrazolo[3,4-d]azepine-8-carboxamide having the structure:

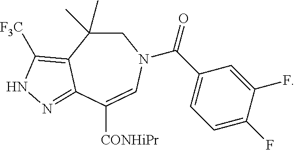

### Pharmaceutical Compositions and Methods of Administration

Administration of FXR modulators as described herein can be in any pharmacological form including a therapeutically effective amount of an FXR modulator alone or in combination with a pharmaceutically acceptable carrier.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of Compound 1, Compound 2, or Compound 3 described herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. Compound 1, Compound 2, or Compound 3 can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes.

Moreover, the pharmaceutical compositions described herein, which include Compound 1, Compound 2, or Compound 3 described herein, can be formulated into any suitable dosage form, including but not limited to, aqueous oral dispersions, liquids, gels, syrups, elixirs, slurries, suspensions, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In some embodiments, Compound 1 is formulated in a tablet dosage form. In some embodiments, Compound 1 is formulated in a capsule dosage form. In some embodiments, Compound 1 is formulated in a suspension dosage form. In some embodiments, Compound 1 is formulated as powder-in-capsule dosage form. In some embodiments, Compound 1 is formulated as a powder-in-bottle for reconstitution as a suspension.

In some embodiments, Compound 2 is formulated in a tablet dosage form. In some embodiments, Compound 2 is formulated in a capsule dosage form. In some embodiments, Compound 2 is formulated in a suspension dosage form. In some embodiments, Compound 2 is formulated as powder-in-capsule dosage form. In some embodiments, Compound 2 is formulated as a powder-in-bottle for reconstitution as a suspension.

In some embodiments, Compound 3 is formulated in a tablet dosage form. In some embodiments, Compound 3 is formulated in a capsule dosage form. In some embodiments, Compound 3 is formulated in a suspension dosage form. In some embodiments, Compound 3 is formulated as powder-in-capsule dosage form. In some embodiments, Compound 3 is formulated as a powder-in-bottle for reconstitution as a suspension.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

Dose administration can be repeated depending upon the pharmacokinetic parameters of the dosage formulation and the route of administration used.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of Compound 1, Compound 2, or Compound 3 and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the Compound 1, Compound 2, or Compound 3 activities disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

### Methods of Dosing and Treatment Regimens

The compounds described herein can be used in the preparation of medicaments for the modulation of FXR, or for the treatment of diseases or conditions that would benefit, at least in part, from modulation of FXR. In addition, a method for treating any of the diseases or conditions described herein in a subject in need of such treatment, involves administration of pharmaceutical compositions containing at least one compound described herein, or a pharmaceutically acceptable salt, or pharmaceutically acceptable solvate or hydrate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from about 10% to about 100%, including, by way of example only, about 10%, about 15%, about 20%, about 25%, about 30%, 40% t 35%, about 40%, about 45%, about 50%, about 55%, about 60%, about 65%, about 70%, about 75%, about 80%, about 85%, about 90%, about 95%, or about 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.01 mg per day to about 5000 mg per day, in some embodiments, about 1 mg per day to about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials, capsules, bottles, or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

### EXAMPLES

All chemicals, reagents, and solvents were purchased from commercial sources when available and used without further purification. Air- and moisture-insensitive reactions were carried out under an ambient atmosphere, mechanical stirred, and monitored by HPLC. Air- and moisture-sensitive reactions were carried out as described in Experimental Data. NMR spectra were recorded with a Bruker Avance III spectrometer using a 5 mm BBFO probe at 400 MHz and 101 MHz for H and 1³C acquisitions, respectively. Chemical shifts were referenced to the residual ¹H solvent signals (CDCl₃, 7.26) and solvent ¹³C signals (CDCl₃, 77.16). Signals are listed as follows: chemical shift in ppm (multiplicity identified as s=singlet, d=doublet, t=triplet, q=quartet, m=multiplet, br=broad; coupling constants in Hz; integration). Mass spectrometry (MS) was performed via electron scatter ionization (ESI) sources.

General HPLC Method Except:

### Column: Agilent Poroshell 120 EC-C18 4.6*100 mm 2.7 μm

Mobile phase: A: 0.1% H₃PO₄/H₂O B: ACN
VWD: 220 nm Temp: 30° C. Flow rate: 1.0 mL/min

### Program Gradient:

| Time | B % |
|---|---|
| 0.00 | 5% |
| 7.00 | 90% |
| 12.00 | 90% |

Gc Method:

### Carrier Gas: N₂

Column: Agilent HP-5 (30 m×0.32 mm, 0.25 μm)

### Injection Temp: 270° C.

### Split Ratio: 50:1

### Control Model: Constant Flow

Flow Rate: 2.0 mL/min

### Injection Volume: 1 μL

### Oven Temperature Program:

Initial 70° C., hold 2 min, 30° C./min to 300° C., hold 3 min

### Run Time: 14 min

### FID Detector Temp: 300° C.

### Intermediate 1: Synthesis of 4-(2-morpholinoethoxy)benzoyl chloride hydrochloride

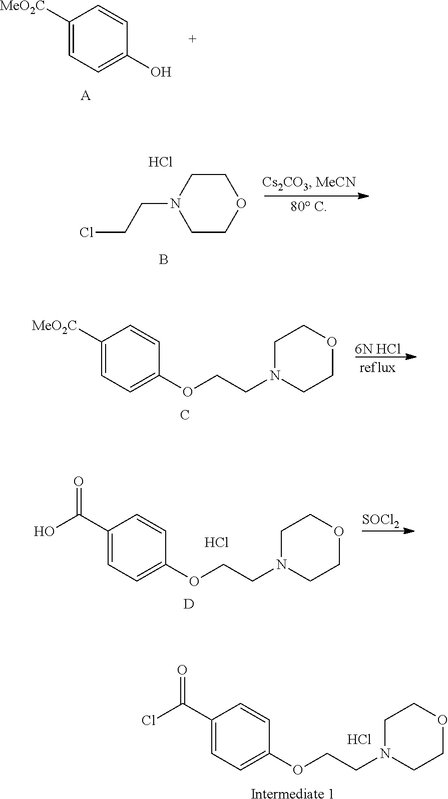

### Step 1: Synthesis of methyl 4-(2-morpholinoethoxy)benzoate (C)

To a 500 L reactor was charged with ACN (180 kg) and Intermediate A (11.6 kg, 76.2 mol, 1.0 eq) under N₂. To the stirred solution was added Cs₂CO₃ (75.2 kg, 230.7 mol, 3 eq) and Intermediate B (17.2 kg, 92.3 mol, 1.2 eq). The mixture was heated to reflux (80-85° C.) for 4 h. The reaction mixture was cooled to room temperature, filtered and rinsed with ACN (50 kg). The volatiles were distilled off at 50-55° C. under vacuum. The residue was partitioned between EA (80 kg) and water (80 kg). The aqueous layer was extracted with EA (25 kg×2). The organic layers were combined, dried over Na₂SO₄ and filtered. The volatiles were evaporated at 50-55° C. under vacuum to afford 18.7 kg of Intermediate C (92% yield) as an off-white solid. ¹H NMR (400 MHz, MeOH-d₄) δ 7.96 (d, J=9.0 Hz, 2H), 7.01 (d, J=8.9 Hz, 2H), 4.20 (t, J=5.5 Hz, 2H), 3.86 (s, 3H), 3.74-3.68 (m, 4H), 2.82 (t, J=5.5 Hz, 2H), 2.63-2.55 (m, 4H). MS: C₁₄H₂₀NO₄ [M+H]⁺ 266.

### Step 2: Synthesis of methyl 4-(2-morpholinoethoxy)benzoic acid hydrochloride (D)

A 20 L flask was charged with 15.5 L of 6 M HCl aqueous solution and Intermediate C (2.25 kg, 8.48 mol) with stirring. The mixture was heated to reflux (110° C.) for 6 h. The mixture was cooled to room temperature and concentrated at 80˜85° C. under vacuum to leave ˜6 L residue. The mixture was cooled to 0-5° C. and stirred at this temp for 30 min. Filtered, and the solid was washed with 2 M aq. HCl (2 L ×2, cooled to 0˜5° C. before use), dried at 70˜80° C. under vacuum for 8 h till water content <0.3 w % (by KF) to afford 2.51 kg of Intermediate D (93% yield) as an off-white solid. ¹H NMR (400 MHz, MeOH-d₄) δ 8.04-7.99 (m, 2H), 7.10 (d, J=8.9 Hz, 2H), 4.88 (s, 4H), 4.53-4.45 (m, 2H), 3.98 (s, 4H), 3.72-3.65 (m, 2H). MS: C₁₃H₁₈NO₄ [M+H]⁺ 252. In total, 23.77 kg of Intermediate D was obtained from 21.25 kg of Intermediate C (2.25 kg×9 batches and 1.0 kg×1 batch) in 93.4% yield.

### Step 3: Synthesis of 4-(2-morpholinoethoxy)benzoyl chloride hydrochloride (Intermediate 1)

A 20 L flask was charged with SOCl₂ (18.55 kg, 3.5 w) and Intermediate D (5.3 kg, 18.4 mol) with stirring. The mixture was stirred at 25˜30° C. for 1 h. The mixture was concentrated at 20˜25° C. under vacuum for 2 h to leave ˜12 kg of residue. PE (10 kg) was added and the mixture was stirred for 10 min. Filtered, and the cake was washed with PE (3 kg) and dried at 45° C. under vacuum for 8 h to afford 5.54 kg of Intermediate 1 (98% yield) as an off-white solid. ¹H NMR (400 MHz, MeOH-d₄) δ 8.04-7.96 (m, 2H), 7.16-7.07 (m, 2H), 4.96 (s, 2H), 4.56-4.46 (m, 2H), 4.07 (dd, J=13.1, 3.4 Hz, 2H), 3.94-3.82 (m, 2H), 3.73-3.65 (m, 2H), 3.61 (d, J=13.1 Hz, 2H). In total, 19.78 kg of Intermediate 1 (Lot #HD 15110716-0471017-16-03) was obtained form 19 kg of Intermediate D in 4 batches in 98% yield.

### Intermediate 2: Synthesis of (2-isopropoxy-2-oxoethyl)zinc(II) bromide

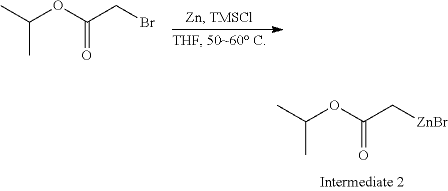

### Step 1: Activation of Zn

A 10 L flask was charged with 4 kg of water and 1.5 kg of Zn powder under agitation. To the suspension was added dropwise 2.5 L of 2.5 M HCl aqueous solution at room temperature over 2 h. The mixture was stirred for 1 h, and then filtered. The cake was washed successively with 3 kg of water, 1.5 kg of IPA and 1.5 kg of MTBE. The wet Zn powder was dried at 60° C. under vacuum for 6 h. The activated Zn powder was kept in N₂ atmosphere.

### Step 2: Synthesis of (2-isopropoxy-2-oxoethyl)zinc(II) bromide (Intermediate 2)

A 20 L flask was evacuated and backfilled with N₂, and activated Zn powder (1.26 kg, 19.2 mol, 2.4 eq) and 10.2 kg of anhydrous THF (KF: 85 ppm) were added under N₂. TMSCl (0.26 kg, 2.4 mol, 0.3 eq) was added to the suspension over 2 min at room temperature. The flask was evacuated and backfilled with N₂. The suspension was stirred at 15˜25° C. for 20 min, heated to 50˜60° C. and stirred for 20 min. A solution of isopropyl 2-bromoacetate (1.45 kg, 8.0 mol, 1.0 eq) in 2.7 kg of THF was added dropwise over 3 h while maintaining the internal temperature between 50˜60° C. The mixture was stirred at this temperature for 0.5 h, and stood for 20 min to settle down the Zn powder. Intermediate 2 was obtained as an olive-green THF solution and kept at 40-50° C. for later use. A sample of THF solution of Intermediate 2 was titrated with 254 mg of 1₂ in 0.5 M LiCl/THF solution to determine its exact concentration.

### Intermediate 3: Synthesis of (E)-isopropyl 2-(4-methoxybenzyl)-4,4-dimethyl-3-(trifluoromethyl)-2,4,5,6-tetrahydropyrazolo[3,4-d]azepine-8-carboxylate

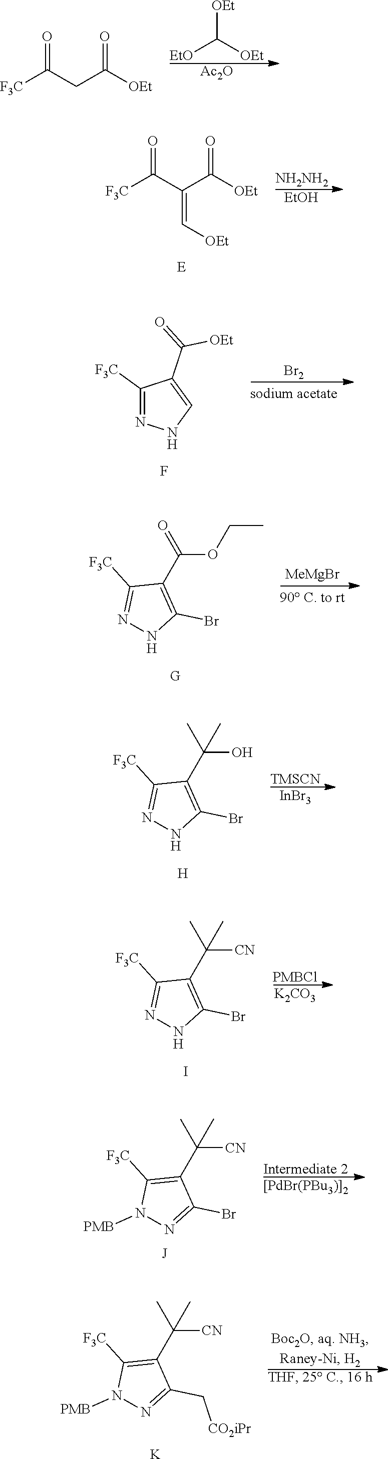

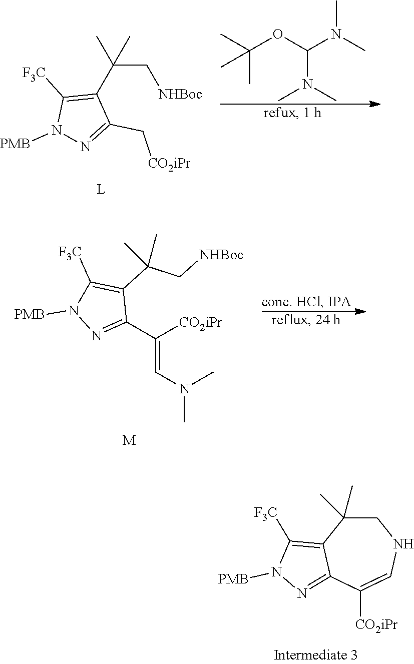

### Step 1: Synthesis of (Z)-ethyl 2-(ethoxymethylene)-4,4,4-trifluoro-3-oxobutanoate (E)

To a solution of ethyl-(4,4,4,-trifluoro)-3-oxobutyrate (25 kg, 135.9 mol, 1 eq.) in acetic anhydride (40 kg, 393 mol, 2.9 eq.), was added triethyl orthoformate (35.7 kg, 241 mol, 1.77 eq.). The reaction mixture was heated at 120° C. for 5 hr. Then, the low-boiling components were removed in rotavapor and the residue was distilled in reduce pressure to give ethyl 2-ethoxymethyllene-4,4,4-trifluoro-3-oxobutytate (E) oil (28.4 kg, 87% yield), which was used without further purification.

### Step 2: Synthesis of ethyl 3-(trifluoromethyl)-1H-pyrazole-4-carboxylate (F)

To a stirred solution of intermediate E (28.2 kg, 117.5 mol, 1 eq.) in EtOH (250 L), was added hydrazine hydrate (85%, 8.3 L, 141 mol, 1.2 eq.) dropwise at 0° C. The reaction mixture was allowed to warm to room temperature and stirred for 8 hour. Then, the mixture was concentrated and the residue was dissolved in 100 L EtOAc. The solution was washed with water, 0.5N HCl and brine. The organic phase was dried over Na₂SO₄ and concentrated to dryness to afford crude yellow product. This crude product washed with warmed EtOAc/PE to get Intermediate F (16.8 kg, 68.7% yield) as a white solid, which was used without further purification.

### Step 3: Synthesis of ethyl 5-bromo-3-(trifluoromethyl)-1H-pyrazole-4-carboxylate (G)

Intermediate F (16.7 kg, 80.2 mol, 1.0 eq.) was dissolved in acetic acid (100 L), added sodium acetate (10.5 kg, 128 mol, 1.6 eq.). To the suspended solution was added a solution of Br₂ (19.3 kg, 120.6 mol, 1.5 eq.). The resulting mixture was stirred at room temperature for 2 hours, and then heated to reflux overnight. The solvent and excess Br₂ were removed in vacuo. The residue was suspended in 150 L ice-water. The white precipitate was collected by filtration and washed with 2×10 L water. The solid was dried in high vacuo to provide Intermediate G (20 kg, yield 86.8%) which was used without further purification.

### Step 4: Synthesis of 2-(5-bromo-3-(trifluoromethyl)-1H-pyrazol-4-yl)propan-2-ol (H)

Intermediate G (20 kg, 69.7 mol, 1.0 eq.) was dissolved in dry THF (100 L), MeMgBr (100 L, 3M in 2-Me-THF, 4.3 eq.) was added dropwise under N₂ at 0° C. The resulting mixture was stirred at 0° C. for 2 hours and then at room temperature overnight. The reaction was cooled to 0° C., quenched with saturated NH₄Cl solution (200 L). The water phase was extracted with AcOEt (100 L ×2), then combined the organic phase and washed with water and brine, dried over Na₂SO₄, filtered and concentrated to afford crude Intermediate H (yellow oil, 18.2 kg). Step 1 through step 4 were repeated two more times (second batch: 18.0 kg, third batch: 18.5 kg). Purification: 54.7 kg crude Intermediate H (combined three batches) was dissolved in 150 L solvent (AcOEt:PE=1:10) and let sit for 2 days at −15° C., then white solid appeared, collected by filtration, washed with PE, dried to get 31.2 kg Intermediate H.

### Step 5: Synthesis of 2-(3-bromo-5-(trifluoromethyl)-1H-pyrazol-4-yl)-2-methylpropanenitrile (I)

InBr₃ was dried in a vacuum oven at 50˜60° C. for 8 h before use. A 1500 L reactor was charged with DCM (250 kg), InBr₃ (2.6 kg, 7.3 mol, 0.1 eq) and TMSCN (21.8 kg, 219.7 mol, 3.0 eq) under N₂. The mixture was heated to 25˜35° C. A solution of Intermediate H (20.0 kg, 73.2 mol, 1.0 eq) in DCM (550 kg) was added dropwise over 2 h at 25˜35° C. The reaction was quenched by charging 850 kg of saturated NaHCO₃ aqueous solution. The mixture was filtered through a celite pad and rinsed with DCM (100 kg). Layers were separated. The aqueous layer was extracted with DCM (300 kg). The combined organic layer was dried over Na₂SO₄, filtered, and the cake was washed with DCM (100 kg). The filtrate was combined with another batch from 6 kg of Intermediate H and concentrated at 30˜40° C. under vacuum to give 29.5 kg of crude Intermediate I as a brown oil. The crude was used into next step without purification. ¹H NMR (400 MHz, CDCl₃) δ 11.72 (s, 1H), 1.79 (s, 6H). MS: C₈H₈BrF₃N₃[M+H]⁺ 282. In total, 27.15 kg of Intermediate H was used in trials and scale-ups and afforded 30.8 kg of Intermediate I.

### Step 6: Synthesis of 2-(3-bromo-1-(4-methoxybenzyl)-5-(trifluoromethyl)-1H-pyrazol-4-yl)-2-methylpropanenitrile (J)

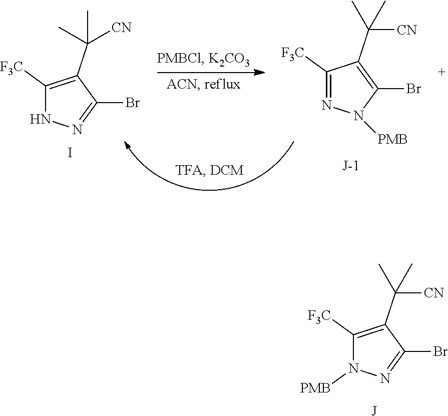

A 500 L reactor was charged ACN (210 kg), crude Intermediate I (29.5 kg, 95.2 mol, 1.0 eq, calculated as theory amount), K₂CO₃ (39.5 kg, 285.8 mol, 3.0 eq) and PMBCl (17.9 kg, 114.6 mol, 1.2 eq) under N₂. The mixture was heated to reflux for 2 h. The mixture was cooled to 30˜40° C., filtered and the wet cake rinsed with ACN (50 kg). The volatiles were distilled off at 40˜50° C. under vacuum to afford a mixture of Intermediates J and J-1 as a brown oil. The residue was dissolved in DCM (12 kg) and filtered. TFA (32 kg) was added to the filtrate and the mixture was stirred for 2 h at 15˜25° C. The volatiles were distilled off at 40˜50° C. under vacuum. The residue was dissolved in ACN (45 kg), and to this were added K₂CO₃ (13.8 kg, 99.8 mol, 1.0 eq), PMBCl (4.5 kg, 28.8 mol, 0.3 eq). The resulting mixture was heated to reflux for 2 h. The mixture was cooled to 30˜40° C., filtered and rinsed with ACN (20 kg). The volatiles were distilled off at 40˜50° C. under vacuum to give a mixture of Intermediates J and J-1. The mixture was dissolved in DCM (12 kg) and filtered. TFA (32 kg) was added to the filtrate and the mixture was stirred for 2 h at 15˜25° C. The volatiles were distilled off at 40-50° C. under vacuum. The residue was dissolved in ACN (45 kg), and to this were added K₂CO₃ (6.0 kg, 43.4 mol, 0.46 eq), PMBCl (1.5 kg, 9.6 mol, 0.1 eq). The result mixture was heated to reflux for 2 h. The mixture was cooled to 30˜40° C., filtered and the wet cake rinsed with ACN (20 kg). The organics were concentrated at 40˜50° C. under vacuum to give crude Intermediate J as a brown oil. The crude oil was purified on silica gel (200-300 mesh) column (EA/PE=1%˜20%) to afford 21.3 kg of Intermediate J (51% yield) as a yellow solid and Intermediate J:Intermediate J-1=98.6:1.4. ¹H NMR (400 MHz, CDCl₃) δ 7.25 (d, J=8.2 Hz, 2H), 6.88 (d, 2H), 5.37 (s, 2H), 3.80 (s, 3H), 1.81 (s, 6H). Totally, 30.8 kg of Intermediate I was used in trials and scale-ups and afforded 22.4 kg of Intermediate J was obtained in 51% yield.

### Step 7: Synthesis of isopropyl 2-(4-(2-cyanopropan-2-yl)-1-(4-methoxybenzyl)-5-(trifluoromethyl)-1H-pyrazol-3-yl)acetate (K)

A 20 L flask was evacuated and backfilled with N₂. [PdBr(t-Bu₃)]2 (350 g, 0.45 mol, 0.3 eq) was added under N₂. The flask was evacuated and backfilled with N₂ again. A degassed solution of Intermediate J (600 g, 1.5 mol, 1.0 eq) in 8.0 kg of anhydrous THF was added and the result solution was heated to 60° C. Intermediate 2 in THF solution (0.32 M, 7.0 L, 2.24 mol, 1.5 eq) was added over 5˜10 min and the mixture was heated to reflux for 0.5 h. The reaction mixture was cooled, and quenched by adding into 12 kg of saturated aqueous NH₄Cl solution. Layers were separated. The aqueous layer was extracted with 8 kg of i-PrOAc. The combined organic layer was washed with brine, dried over Na₂SO₄ and filtered. The volatiles were removed at 40˜50° C. under vacuum. The residue was extracted with 12 kg of heptane at 80° C. for 30 min, cooled to 50° C. and the upper clear solution was decanted. The residue was treated with 2 kg of heptane by repeating the same procedure. The heptane solutions were combined and cooled to 0˜5° C. over 4 h, stirred at this temperature for 1 h and the solid precipitated out was collected by filtration. The solid was combined with another 3 batches from 1.8 kg of Intermediate J and dried at 50° C. under vacuum to afford 1.55 kg of Intermediate K (61.3% yield) as a yellow solid. ¹H NMR (400 MHz, CDCl₃) δ 7.07 (d, J=8.7 Hz, 2H), 6.86 (d, J=8.7 Hz, 2H), 5.30 (s, 2H), 4.95 (s, 1H), 3.94 (s, 2H), 3.78 (s, 3H), 1.78 (s, 6H), 1.21 (d, J=6.3 Hz, 6H). MS: C₂₁H₂₅F₃N₃O₃, [M+H]⁺ 424. In total, 7.25 kg of Intermediate J was used in trials and scale-ups and afforded 4.62 kg of Intermediate K in 60.5% yield.

### Step 8: Synthesis of isopropyl 2-(4-(1-(tert-butoxycarbonylamino)-2-methylpropan-2-yl)-1-(4-methoxybenzyl)-5-(trifluoromethyl)-1H-pyrazol-3-yl)acetate (L)

Raney-Ni (2.4 kg, 3 weight of Intermediate K) was washed with IPA (2 L ×3) in a 20 L flask, then to the flask were added a solution of Intermediate K (0.8 kg, 1.9 mol, 1.0 eq) in THF (4 L, 5 v), IPA (8 L, 10 v), Boc₂O (1.03 kg, 4.7 mol, 2.5 eq) and aqueous 25 w % ammonia solution (80 mL, 0.1 v). The mixture was stirred under 1 atm. H₂ at 25-30° C. for 16 h. The catalyst was carefully removed by filtration and washed with THF (5 L). The filtrate was concentrated under vacuum at 40° C. to afford 1.1 kg of crude. The residue was slurried in PE (1.1 L, 1 v of the crude) at 5-10° C. for 4 h. The solid was collected by filtration, washed with PE (0.5 L ×2) and dried under vacuum at 25° C. for 3 h to give 0.61 kg of Intermediate L (61.8% yield) as a white solid. In total, 4.62 kg of Intermediate K was used in 4 batches and afforded 4.13 kg of Intermediate L. The mother liquid of 4 batches from 4.62 kg of Intermediate K was recovered by purification on silica gel column (EA/PE=1%-10%) and was slurried in PE (2 L) to afford 0.4 kg of Intermediate L. A total of 4.53 kg of Intermediate L was obtained in 78.7% yield. 1H NMR (400 MHz, CDCl₃) δ 7.05 (d, J=8.6 Hz, 2H), 6.85 (d, J=8.7 Hz, 2H), 5.28 (s, 2H), 4.96 (dt, J=12.5, 6.3 Hz, 1H), 4.57 (s, 1H), 3.77 (s, 3H), 3.72 (s, 2H), 3.26 (d, J=6.2 Hz, 2H), 1.38 (s, 9H), 1.34 (s, 6H), 1.20 (d, J=6.3 Hz, 6H).

MS: C26H37F3N3O5 [M+H]+ 528.

### Step 9: Synthesis of (Z)-isopropyl 2-(4-(1-(tert-butoxycarbonylamino)-2-methylpropan-2-yl)-1-(4-methoxybenzyl)-5-(trifluoromethyl)-1H-pyrazol-3-yl)-3-(dimethylamino)acrylate (M)

A suspension of Intermediate L (4.53 kg, 8.5 mol, 1.0 eq.) and Boc₂O (0.46 kg, 2.1 mol, 0.25 eq) in Bredereck's reagent (tert-butoxy bis(dimethylamino)methane) (13.5 L, 3 V) under N₂ was heated to reflux for 1 h. The mixture was cooled to room temperature, diluted with DCM (65 L), washed with 5 w % aq. citric acid (20 L ×4) and 13 w % brine (20 L ×2), dried over Na₂SO₄, filtered and concentrated at 40˜50° C. under vacuum to give 5.1 kg of crude Intermediate M as a yellow oil. The crude was used into next step without purification. ¹H NMR (400 MHz, CDCl₃) δ 7.53 (s, 1H), 7.26 (d, J=8.5 Hz, 2H), 6.80 (d, J=8.6 Hz, 2H), 5.15 (d, J=14.5 Hz, 1H), 4.97 (dt, J=12.4, 6.2 Hz, 1H), 4.88 (d, J=14.5 Hz, 1H), 4.57 (s, 1H), 3.77 (s, 3H), 3.26 (qd, J=13.6, 6.2 Hz, 2H), 2.87 (s, 3H), 1.80 (d, J=39.5 Hz, 3H), 1.38 (s, 9H), 1.29 (s, 3H), 1.21 (s, 3H), 1.19 (d, J=6.2 Hz, 3H), 1.08 (d, J=6.2 Hz, 3H). MS: C₂₉H₄₂F₃N₄O₅[M+H]⁺ 583.

### Step 10: Synthesis of (E)-isopropyl 2-(4-methoxybenzyl)-4,4-dimethyl-3-(trifluoromethyl)-2,4,5,6-tetrahydropyrazolo[3,4-d]azepine-8-carboxylate (Intermediate 3)

To a solution of crude Intermediate M (1.02 kg, 1.75 mol, 1.0 eq) in iPrOH (15.3 L, 15 v) was added concentrated HCl in water (510 mL, 0.5 v). The resulting mixture was heated to reflux (80˜84° C.) for 24 h. The solution was cooled to 40° C. and concentrated under vacuum at 40-45° C. to remove solvent. The residue was dissolved in DCM (10 L), washed with saturated NaHCO₃ (5 L) and brine (5 L), dried over Na₂SO₄ and filtered. The filtrate was combined with another 4 batches from 4.08 kg of Intermediate M and concentrated under vacuum at 35-40° C. to give crude Intermediate 3 (0.66 kg). The crude product was dissolved in EA (4.3 L) with heating to 40° C. and then PE (8.6 L) was added over 2 min. The solution was stirred at 15˜25° C. for 1 h and then at 0-5° C. for 30 min. The solid was collected by filtration and washed with PE/EA (v/v=5/1, 3 L ×2). The wet cake was dried under vacuum at 50° C. to afford 2.45 kg of Intermediate 3 (64% yield) as a white solid. The mother liquid was purified on silica gel (200˜300 mesh) column with EA/PE=1/2 and then crystallized from EA/PE (1/2, 2 L) to afford 315 g of Intermediate 3. In total, 2.77 kg of Intermediate 3 was obtained in 72% yield. ¹H NMR (400 MHz, CDCl₃) δ 7.46 (d, J=8.2 Hz, 1H), 6.94 (d, J=8.7 Hz, 2H), 6.76 (d, J=8.7 Hz, 2H), 5.55-5.44 (m, 1H), 5.28 (s, 2H), 5.11 (hept, J=6.2 Hz, 1H), 3.75 (s, 3H), 3.05 (d, J=4.7 Hz, 2H), 1.35 (s, 6H), 1.27 (d, J=6.3 Hz, 6H). ¹³C NMR (101 MHz, CDCl₃) δ 166.51 (s), 158.90 (s), 145.06 (s), 138.17 (s), 135.91 (s), 135.55 (s), 128.89 (d, J=15.4 Hz), 125.24 (s), 123.58 (s), 120.91 (s), 113.73 (s), 91.52 (s), 77.33 (d, J=11.8 Hz), 77.07 (s), 76.75 (s), 67.35 (s), 59.45 (s), 55.73 (s), 55.18 (s), 36.11 (s), 25.23 (s), 22.12 (s). MS: C₂₂H₂₇F₃N₃O₃ [M+H]⁺ 438.

### Example 1: Synthesis of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2) and its HCl Salt

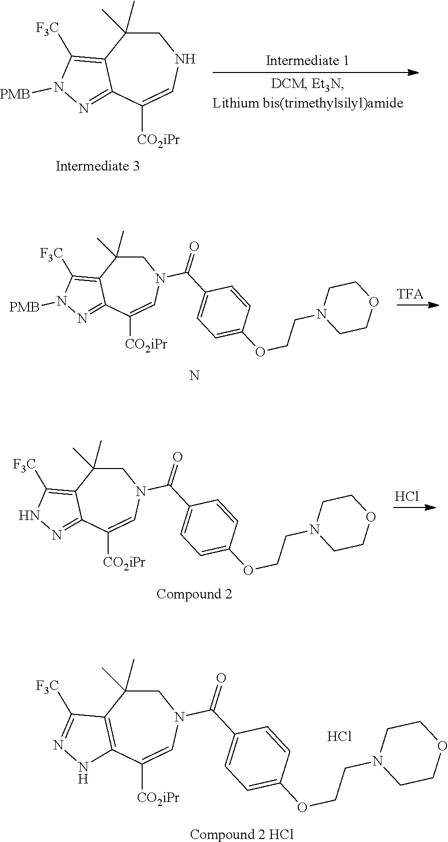

### Step 1: Synthesis of (E)-isopropyl 2-(4-methoxybenzyl)-4,4-dimethyl-6-(4-(2-morpholinoethoxy)benzoyl)-3-(trifluoromethyl)-2,4,5,6-tetrahydropyrazolo[3,4-d]azepine-8-carboxylate (N)

To a suspension of 4-(2-morpholinoethoxy)benzoyl chloride hydrochloride (840 g, 2.74 mol, 1.2 eqs) in DCM (15 L, 15 vol) at 20-25° C. under nitrogen was added triethylamine (414 mL, 2.97 mol, 1.3 eqs). Additional DCM (3 L, 3 vol) was charged as a reagent rinse and the mixture was stirred to dissolution. Isopropyl 2-(4-methoxybenzyl)-4,4-dimethyl-3-(trifluoromethyl)-2,4,5,6-tetrahydropyrazolo[3,4-d]azepine-8-carboxylate (1000 g, 2.29 mol 1 eq) was charged followed by DCM (3 L, 3 vol) as a reagent rinse. The reaction solution was cooled to 0-5° C. and lithium bis(trimethylsilyl)amide (1M in THF/ethylbenzene) (4.25 Kg, 5.26 mol, 2.3 eqs) was added drop-wise over at least 1 hour maintaining the internal temperature at 0-10° C. The reaction mixture was stirred at this temperature for 30 mins and reaction progression was checked by HPLC. The reaction mixture was quenched by addition of saturated aqueous ammonium chloride solution (10-15 L, 10-15 vol) maintaining the internal temperature at 0-10° C. The internal temperature was adjusted to 20-25° C. and purified water (5 L, 5 vol) was added rapidly. The mixture was stirred for 10 mins then allowed to settle for at least 10 mins. The upper aqueous layer was removed and the organic layer was washed again with purified water (10 L, 10 vol). The mixture was stirred for 10 mins then allowed to settle for at least 10 mins. The upper aqueous layer was removed and the organic layer was concentrated to dryness.

### Step 2: Synthesis of (E)-isopropyl 4,4-dimethyl-6-(4-(2-morpholinoethoxy)benzoyl)-3-(trifluoromethyl)-2,4,5,6-tetrahydropyrazolo[3,4-d]azepine-8-carboxylate (Compound 2)

To crude isopropyl 2-(4-methoxybenzyl)-4,4-dimethyl-6-(4-(2-morpholinoethoxy)benzoyl)-3-(trifluoro-methyl)-2,4,5,6-tetrahydropyrazolo[3,4-d]azepine-8-carboxylate (1533 g, 2.29 mol, 1 eq) was charged DCM (10 L, 10 vol) and the mixture was stirred to dissolution at 20-25° C. The solution was concentrated to ca. 3 L (3 vols) under vacuum at 40-45° C. Trifluoroacetic acid (6 L, 142.6 mol, 62 eqs) was added and excess DCM was removed under vacuum at 40-45° C. The solution rapidly turned to a dark purple color from orange. The reaction mixture was then stirred at 40-45° C. at atmospheric pressure for at least 40 mins. The reaction mixture was concentrated to dryness in vacuo at 40-45° C. then redissolved in DCM (10 L, 10 vols). Saturated aqueous sodium bicarbonate solution (20 L, 20 vols) was slowly charged with vigorous stirring to the DCM product solution. Upon addition the resultant yellow mixture was stirred for at least 40 mins to ensure the internal pH had stabilized. The contents were allowed to settle and the neutralized upper aqueous phase (pH >7) was removed. The organic phase was washed with water (10 L, 10 vols) then concentrated in vacuo at 40-45° C. to ca. 3 L (3 vols). The solution was cooled to 20-25° C. and diisopropyl ether (20 L, 20 vols) was slowly charged over at least 30 mins with vigorous stirring. The resultant pale yellow suspension was stirred at 20˜25° C. for at least 12 hours. The precipitate was isolated by filtration and the cake washed with diisopropyl ether (at 20-25° C.) (2×5 L, 2×5 vols). The filter cake was pulled dry for at least 2 hours and the solid was dried in a vacuum oven at 40° C. to constant weight. An off-white solid was afforded (1.17 kg, 93% yield over 2 steps).

### Step 3: Synthesis of (E)-isopropyl 4,4-dimethyl-6-(4-(2-morpholinoethoxy)benzoyl)-3-(trifluoromethyl)-2,4,5,6-tetrahydropyrazolo[3,4-d]azepine-8-carboxylate hydrochloride (Compound 2-HCl)

Isopropyl 4,4-dimethyl-6-(4-(2-morpholinoethoxy)benzoyl)-3-(trifluoromethyl)-2,4,5,6-tetrahydropyrazolo-[3,4-d]azepine-8-carboxylate (1.17 kg, 2.13 mol) was suspended in TBME (129 L, 110 vols) and the suspension warmed up to 45° C. to obtain a cloudy pale yellow solution. The contents were cooled to 20-25° C. and hydrogen chloride (2M in diethyl ether) (1.17 L, 2.34 mol, 1.1 eqs) was added drop-wise over ca. 20 min to form a thick white suspension. The suspension was stirred for 2-18 hours at 25° C. and was isolated by filtration. The cake was washed with TBME (2×6 L, 2×5 vols) and pulled dry for at least 2 hours and oven dried at 40° C. prior to re-slurry in isopropyl acetate (IPAC). The crude salt was re-slurried in IPAC (35 L, 30 vols) at 40-45° C. for at least 90 mins. The contents were adjusted to 20-25° C. and the precipitate was isolated by filtration and washed with IPAC (2×5 L, 2×4.3 vols) to afford a white solid (1.07 kg, 86% yield). ¹H NMR (400 MHz, DMSO) δ 13.09 (s, 1H), 11.81 (s, 1H), 7.91 (s, 1H), 7.62 (d, J=8.7 Hz, 2H), 7.15 (d, J=8.7 Hz, 2H), 5.02 (dt, J=12.4, 6.2 Hz, 1H), 4.56 (d, J=4.4 Hz, 2H), 4.03-3.81 (m, 6H), 3.57 (s, 2H), 3.48 (d, J=12.0 Hz, 2H), 3.21 (d, J=7.6 Hz, 2H), 1.32 (s, 6H), 1.17 (d, J=6.2 Hz, 6H). ¹³C NMR (101 MHz, DMSO) δ 169.81 (s), 165.15 (s), 160.27 (s), 143.02 (s), 133.92 (s), 131.30 (s), 126.03 (s), 124.50 (s), 123.71 (s), 121.04 (s), 114.67 (s), 102.02 (s), 68.91 (s), 63.06 (s), 62.56 (s), 54.91 (s), 54.55 (s), 51.56 (s), 40.15 (s), 39.94 (s), 39.73 (s), 39.52 (s), 39.31 (s), 39.10 (s), 38.89 (s), 35.13 (s), 26.68 (s), 21.34 (s). ¹⁹F NMR (376 MHz, DMSO) δ −56.06 (s). MS: C₂₇H₃₃F₃N₄O₅, [M+H]⁺ 551.

### Intermediate 4: Synthesis of 3-fluoro-4-(2-morpholinoethoxy)benzoic hypochlorous anhydride hydrochloride

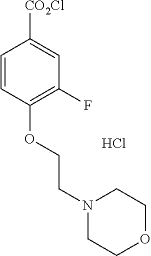

The title compound was synthesized as described in Intermediate 1 using methyl 3-fluoro-4-hydroxybenzoate as the starting material.

### Example 2: Synthesis of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3)

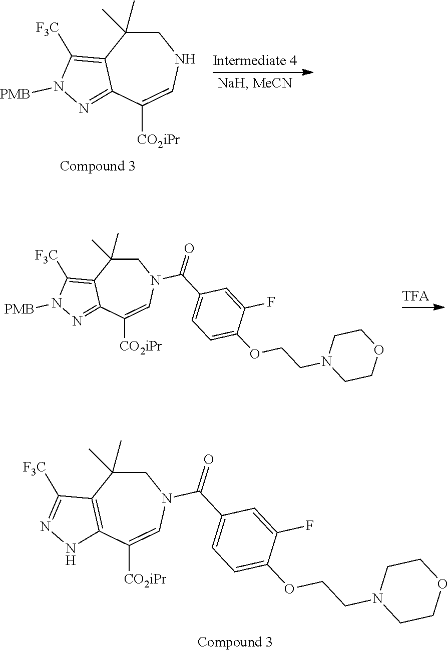

In a similar manner as described in Example 1, (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3) was prepared starting from Intermediate 3 and 3-fluoro-4-(2-morpholinoethoxy)benzoyl chloride as a white solid. LCMS m/z: 569.3 [M+H]⁺.

### Example 3: Synthesis of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1)

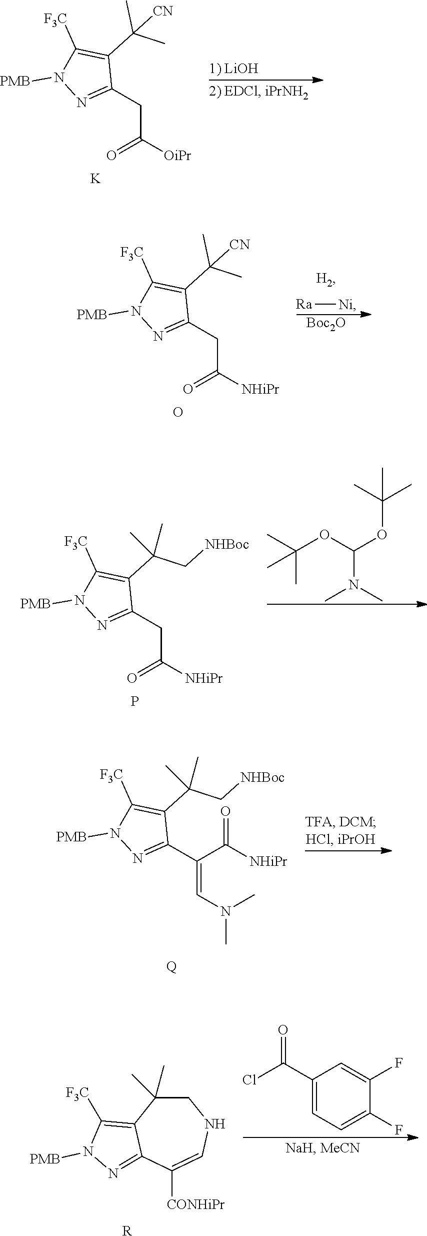

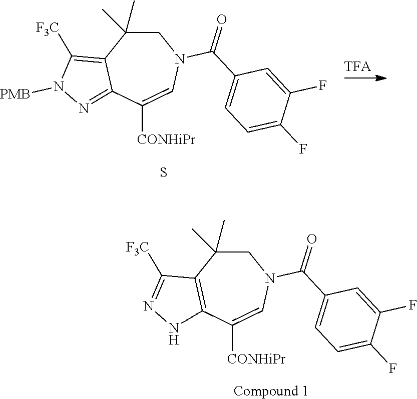

Compound 1 was prepared starting from Intermediate K as described in the scheme above. LCMS m/z: 457.6 [M+H]⁺.

### Example 4: FXR Agonist Assay

Starting from 3.33 mM of compound in DMSO solution, a 10-point 3-fold serial dilution was made by diluting 5 μL of compound into 10 μL of DMSO. The serially diluted compound was then diluted 1:33 into DMEM. This medium was then diluted ten-fold into the culture medium with the cells (10 μL/well). All concentration points are assayed in duplicate. Plates were incubated at 37° C. for 20 hours. After the incubation, 20 μL of culture medium were removed from each well and mixed with 50 μL of assay solution (Pierce™ Gaussia Luciferase Flash Assay Kit). The luminescence was measured immediately after addition of the Luc substrate with an Envision microplate reader. The raw data was uploaded to CDD and dose-response curves were generated using the Levenberg-Marquardt algorithm integrated into CDD. A negative control DMSO is included on each plate and used to normalize the data with the CDD built-in normalization function. Compounds 1, 2 and 3 all had EC₅₀ values less than 500 nM.

The examples and embodiments described herein are for illustrative purposes only and in some embodiments, various modifications or changes are to be included within the purview of disclosure and scope of the appended claims.

1.-44. (canceled)

45. A process for the preparation of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2): 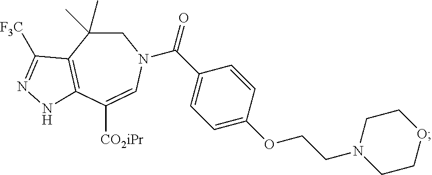
comprising contacting a compound with the structure (Compound N): 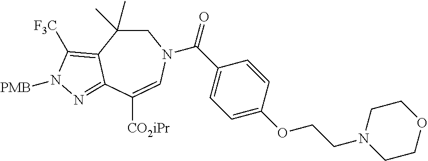
with acid followed by base.

46. The process of claim 45, wherein the acid to produce (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2) is trifluoracetic acid.

47. The process of claim 45, wherein the compound with the structure (Compound N): 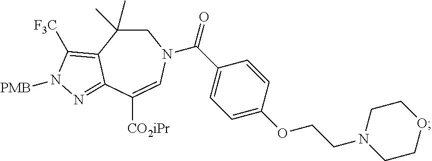
is prepared by a process comprising contacting a compound with the structure (Intermediate 3): 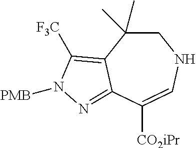
with base and 4-(2-morpholinoethoxy)benzoyl chloride hydrochloride in the presence of a solvent.

48. The process of claim 45, wherein the base is sodium hydride, sodium bis(trimethylsilyl)amide, or lithium bis(trimethylsilyl)amide.

49. The process of claim 47, wherein the compound having the structure of: 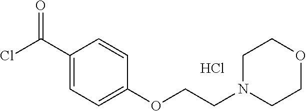 [SMILES: O=C(Cl)c1ccc(OCCN2CCOCC2)cc1 ; Cl]
is prepared by a process comprising contacting a compound with the structure (Compound D): 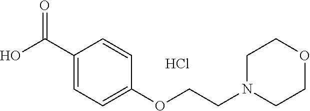 [SMILES: O=C(O)c1ccc(OCCN2CCOCC2)cc1 ; Cl]
with thionyl chloride in the presence of a solvent.

50. The process of claim 49, wherein the compound having the structure: 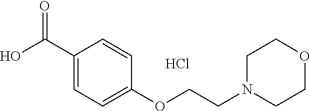 [SMILES: O=C(O)c1ccc(OCCN2CCOCC2)cc1 ; Cl]
is prepared by a process comprising contacting a compound with the structure (Compound C): 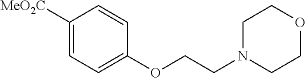 [SMILES: COC(=O)c1ccc(OCCN2CCOCC2)cc1]
with concentrated hydrochloric acid.

51. The process of claim 50, wherein the compound having the structure: 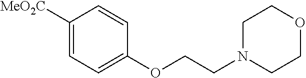 [SMILES: COC(=O)c1ccc(OCCN2CCOCC2)cc1]
is prepared by a process comprising a compound with the structure: 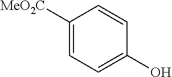 [SMILES: COC(=O)c1ccc(O)cc1]
with 4-(2-chloroethyl)morpholine hydrochloric acid and cesium carbonate in the presence of a solvent.

52. The process of claim 45 further comprising treating a compound with the structure (Compound 2): 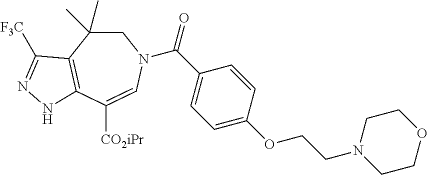
with hydrochloric acid in the presence of a solvent to produce (E)-isopropyl-4,4-dimethyl-6-(4-(2-morpholinoethoxy)benzoyl)-3-(trifluoromethyl)-1,4,5,6-tetrahydropyrazolo[3,4-d]azepine-8-carboxylate hydrochloride having the structure (Compound 2 HCl):

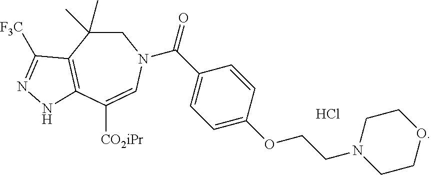

53. A process for the preparation of a compound with the structure (Intermediate 3): 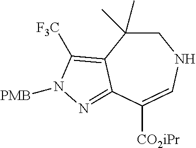
comprising contacting a compound with the structure (Compound M): 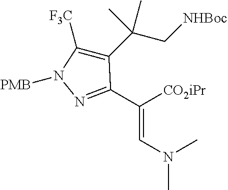
with acid in the presence of a solvent.

54. The process of claim 53, wherein the acid is hydrochloric acid.

55. The process of claim 53, wherein the compound with the structure (Compound M): 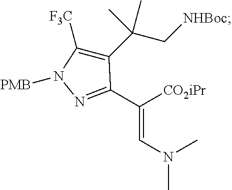
is prepared by a process comprising contacting a compound with the structure (Compound L): 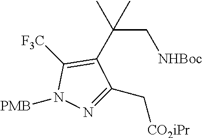
with tert-butoxy-bis(dimethylamino)methane.

56. The process of claim 55, wherein the compound with the structure (Compound L): 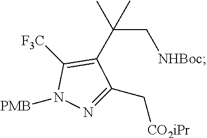
is prepared by a process comprising contacting a compound with the structure (Compound K-Intermediate 2): 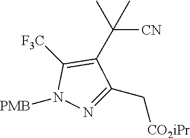
with Raney nickel, di-tert-butyl dicarbonate, and aqueous 25 weight % ammonia solution, under a hydrogen atmosphere in the presence of a solvent.

57. The process of claim 56, wherein the compound with the structure (Compound K-Intermediate 2): 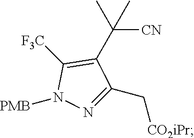
is prepared by a process comprising contacting a compound with the structure (Compound J): 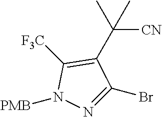
with (2-isopropoxy-2-oxoethyl)zinc(II) bromide and bromo(tri-tert-butylphosphine)palladium(I) dimer in the presence of a solvent.

58. The process of claim 57, wherein the compound with the structure: 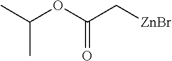
is prepared by a process comprising contacting a compound with the structure: 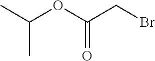
with zinc and chlorotrimethylsilane.

59. The process of claim 57, wherein the compound with the structure (Compound J): 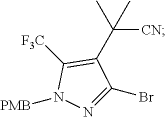
is prepared by a process comprising contacting a compound with the structure (Compound I): 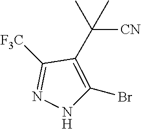
with 4-methoxybenzyl chloride and potassium carbonate in the presence of a solvent.

60. The process of claim 59, wherein the compound with the structure (Compound I): 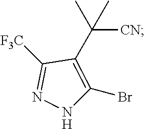
is prepared by a process comprising contacting a compound with the structure (Compound H): 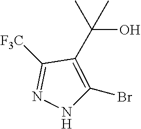
with trimethylsilyl cyanide and indium(III) bromide in the presence of a solvent.

61. The process of claim 60, wherein the compound with the structure (Compound H): 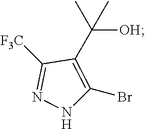
is prepared by a process comprising contacting a compound with the structure (Compound G): 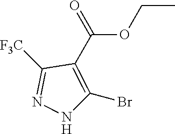
with methyl magnesium bromide in the presence of a solvent.

62. The process of claim 61, wherein the compound with the structure (Compound G): 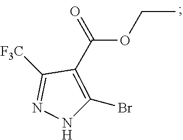
is prepared by a process comprising contacting a compound with the structure (Compound F): 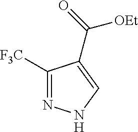 [SMILES: CCOC(=O)c1c[nH]nc1C(F)(F)F]
with bromine and sodium acetate in acetic acid.

63. The process of claim 62, wherein the compound with the structure (Compound F): 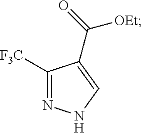 [SMILES: CCOC(=O)c1c[nH]nc1C(F)(F)F]
is prepared by a process comprising contacting a compound with the structure (Compound E): 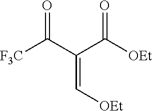 [SMILES: CCO/C=C(\C(=O)OCC)C(=O)C(F)(F)F]
with hydrazine hydrate in the presence of a solvent.

64. The process of claim 63, wherein the compound with the structure (Compound E): 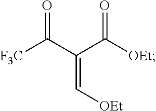 [SMILES: CCO/C=C(\C(=O)OCC)C(=O)C(F)(F)F]
is prepared by a process comprising contacting a compound with the structure: 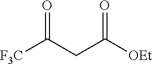 [SMILES: CCOC(=O)CC(=O)C(F)(F)F]
with triethyl orthoformate and acetic anhydride.
